(19) United States
(12) Patent Application Publication
(10) Pub. No.: US 2002/0173538 A1
(43) Pub. Date: Nov. 21, 2002

(54) Method for sensitizing cancer cells to cancer therapies with a mevalonate-reducing compound

(76) Inventor: Ming-Shi Shiao, Taipei (TW)

Correspondence Address: Kent H. Cheng, Esq., Cohen, Pontani, Lieberman & Pavane, Suite 1210, 551 Fifth Avenue, New York, NY, 10176, US

(21) Appl. No.: 09/897,627

(22) Filed: Jul. 2, 2001

Related U.S. Application Data
(60) Provisional application No. 60/215,642, filed on Jun. 30, 2000.

(51) Int. Cl. A61K 31/337; A61K 31/366

(52) U.S. Cl. 514/449; 514/460

(57) ABSTRACT

The present invention provides a pharmaceutical kit useful for treating cancer cells in a mammal. Using this kit, the cancer cells are first sensitized by administering to the mammal an agent which comprises an effective amount of a HMG-CoA reductase inhibitor. The sensitized cancer cells are then treated with an anti-cancer agent which reduces the survival or growth of the cancer cells.

### BACKGROUND OF THE INVENTION

1. Field of the Invention

The present invention relates to a method for treating cancers. More particularly, the invention pertains to a method of sensitizing cancer cells to cancer therapies by administrating sequentially a mevalonate reducing compound and a cancer treating agent, whereby said mevalonate reducing compound causes the cancers cells arrested at a specific stage of the cell cycle to render the cancer cells more susceptible to said cancer treating agent which is subsequently administrated.

2. DESCRIPTION OF THE RELATED ART

It is well known in the art that different forms of cancer, although sharing certain common characteristics, demand different treatment. For example, paclitaxel has been known to be an effective cancer-treating drug for breast cancer, ovarian cancer, non-small cell lung cancer and Kaposi's sarcoma, but it has not been shown to have therapeutic benefit for treating liver cancer. It is also known that many cancer-treating agents have toxic effects on cancer cells but also exert similar effects on normal cells and their usefulness is limited because patients may not be able to tolerate the dosage needed for effective killing of cancer cells.

Although there have been efforts to combine different anti-cancer agents to augment the therapeutic effects while keeping the side-effects tolerable, to applicant's knowledge, no generally applicable approach is available to guide the combination use of different anti-cancer agents under various situations with respect to different forms of cancer.

The present invention provides a general approach to sequentially use two or more anti-cancer drugs in cancer treatment by making reference to various stages of the cell cycle.

### SUMMARY OF THE INVENTION

In accordance with the present invention, a mevalonate-depleting compound, such as lovastatin, simvastatin, provastatin or atorvastatin, can be used to arrest cancer cells at G0/G1 phase of the cell cycle, sensitizing the cancer cells to subsequently applied anti-cancer drug which arrests the cancer cells at a different phase of the cell cycle, such as the G2/M phase. Furthermore, according to the present invention, the mevalonate-depleting compound does not significantly render normal cells more susceptible to anti-cancer drug's toxic activities because normal cells are better equipped to cope with mevalonate depletion than cancer cells. Thus, the present invention provides a more effective cancer treatment method with a higher ratio of therapeutic effect to side effect.

As one embodiment of the present invention, significant cancer treatment results are achieved in the nude mice (BALC/c-nu/nu mice) inoculated with Hep 3B/T2 cells.

As another embodiment of the present invention, cancer prevention and treatment can be realized by calculated sequential use of one type of food supplements containing a mevalonate-reducing compound, such as monacolin K, followed by another type of food supplements containing an apoptotic compound, such as extracts from Ganoderma.

One of the objectives of the present invention is to provide a method for combining more than one compound in cancer treatment. This objective is achieved by selecting compounds which work at different stages of the cell cycle and timing the sequential treatments according to the various phrases of the cell cycle.

Other objects and features of the present invention will become apparent from the following detailed description considered in conjunction with the accompanying drawings. It is to be understood, however, that the drawings are designed solely for purposes of illustration and not as a definition of the limits of the invention, for which reference should be made to the claims.

### DETAILED DESCRIPTION OF THE PRESENTLY PREFERRED EMBODIMENTS

Hepatocellular carcinoma (HCC) is one of the leading cause of death among cancer patients, particularly in Asia (Okuda, K. 1996. Jpn. J. Cancer Chemother., 23(9): 1105-1115; Beasley, R. P., et. a9 ., 1981. Lancet, 2: 1129-1133; Ince, N. and J. R. Wands. 1999.N. Engl. J. Med., 340: 798-799.). The high mortality of HCC is due to poor diagnosis and lack of effective anticancer therapy. Mevalonate is an obligatory intermediate in the mevalonate pathway and the multiple mevalonate-demanding routes, which are vital for diverse cell function. Many studies have shown that reduction in mevalonate formation results in a significant decrease of cholesterol synthesis and a less or various extents of reduction in protein isoprenylation including Ras and G-proteins. Further reduction of mevalonate formation causes cell cycle arrest (Jakobisiak, M., et. al.,. 1991. Proc. Natl. Acad. Sci. USA, 88: 3628-3632; Srikant, C. B. 1995. Biochem. Biophys. Res. Comm., 209(2): 400-406; Jones, K. D., et. al., 1994. Biochem. Biophys. Res. Comm., 250: 1681-1687; Sumi, S., et. al., 1992. Gastroenterology, 103: 982-989; Rao, S., et. al., 1999. Proc. Natl. Acad. Sci. USA, 96: 7797-7802.), inhibition of DNA synthesis, and cell apoptosis (7hang, F. L. and P. J. Casey. 1996. Annu. Rev. Biochem., 65: 241-269; Keyomarsi, K., L. et. al., 1991. Cancer Res., 51: 3602-3609; Kawata, S., E. et. al., 2001. Brit. J. Cancer, 84: 886-891). The mevalonate pathway, which is essential for cell function, may be disturbed in cancer cells in at least two ways. The first is that the demand of mevalonate by those mevalonate-dependent pathways may be perturbed in cancer cells. Another one is that mevalonate-perturbed cancer cells may become more sensitive to another anticancer agent. II is also possible that particular types of cancer may respond to mevalonate deprivation by both. Our study indicated that HCC is among the human cancers that the above-mentioned situation may apply.

We have elucidated the differences in responses (particularly in cell viability and apoptosis) of cultured human hepatoma cells to the multiple mevalonate-demanding routes when mevalonate formation is reduced progressively. Lovastatin, an HMG-CoA reductase inhibitor and cholesterol-lowering drug, was used to reduce mevalonate pool. This statin is more lipophilic (than several other statins) and shows very high liver-specificity, a property crucial to the treatment of human HCC according to our working hypothesis. The concentration of this statin is higher in the liver than in other tissues and organs. Besides, the statins are usually low in toxicity.

Human hepatoma cell lines Hep G2 and Hep 3 were chosen as the in vitro models of hepatocytes and hepatoma, respectively. Hep G2 cell line is derived from the hepatocyte-like human hepatoblastoma whose cholesterol and lipoprotein metabolism remains normal. The sensitivity of Hep G2 to lovastatin was found very similar to that of isolated mouse hepatocytes. Hep 3B cells are p53- and bcl-2 deficient, but can express the MDR-1 gene. Most importantly, Hep 3 cells produce tumor in nude mice (BALB/c-nu/nu) if inoculated to animals.

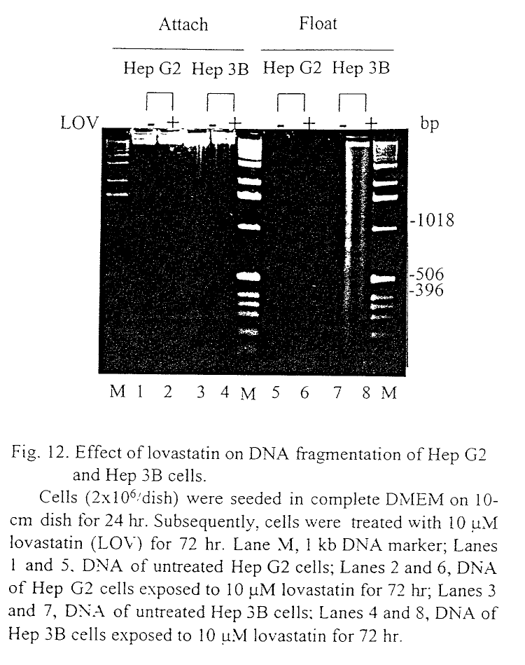

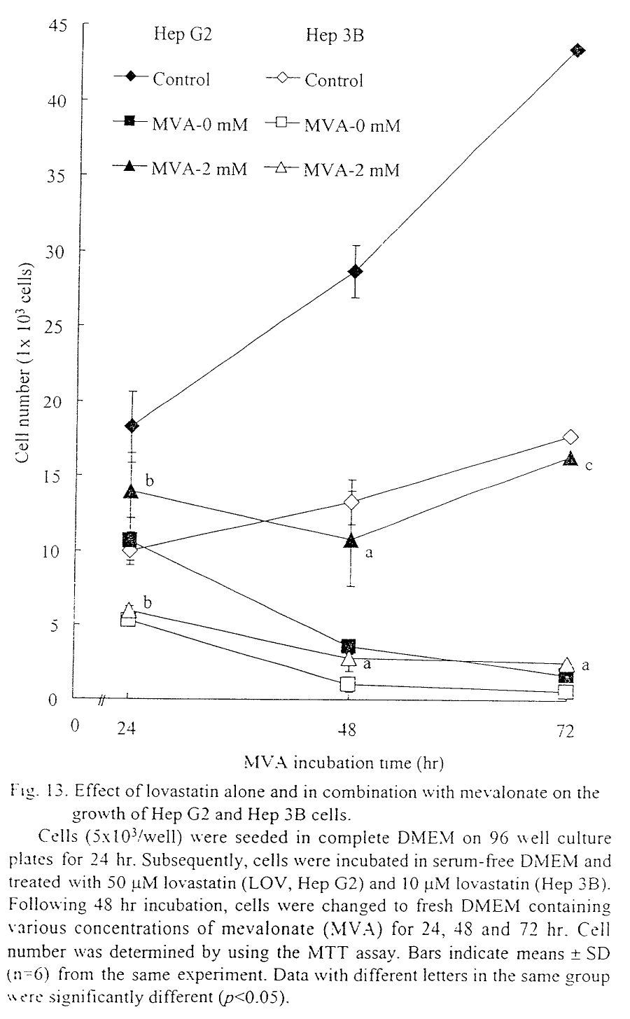

As shown on Table 4, the viabilities of Hep G2 and Hep 3B cells were decreased by lovastatin in a time- and dose-dependent manner. In 48 hr and 72 hr treatment, the IC₅₀ values of lovastatin for Hep G2 cells were 97.2±2.1 M and 62.3±3.7 M, respectively. The HCC-like Hep 3B cells were more sensitive to lovastatin (the IC₅₀ values were 63.6±7.1 M at 48 hr and 6.8±1.3 M at 72 hr, respectively). The great differences in IC₅₀ values indicated that the HCC-like Hep 3B cells were more sensitive to the reduction of mevalonate pool. Mevalonate supplementation significantly reversed lovastatin-inhibited cell growth in Hep G2 cells (Table 6 and 7). Lovastatin-treated Hep 3B cells responded to mevalonate treatment very poorly. Lovastatin inhibited cholesterol biosynthesis in Hep G2 and Hep 3 cells, as determined by incorporation of [2, 3H]-acetate into cholesterol, to a very similar extent. After a prolonged treatment with lovastatin, Hep G2 cells started to recover cholesterol biosynthetic capability. However, cholesterol synthesis in Hep 3B cells remained highly suppressed (>90%). Cell cycle determination by using the flow cytometry technique showed that lovastatin arrested Hep G2 and Hep 3B cells at G0/G1 (FIG. 11), as that reported in other cells studied. Sub-G1 population and DNA fragmentation (as markers of cell apoptosis) in Hep 3B cells were observed in a higher percentage and lower dose of lovastatin treatment than those in Hep G2 cells treated in the same condition (FIG. 12). It suggested that the HCC-like Hep 3B cells were significantly more sensitive to the reduction of cellular mevalonate compartment and became apoptosis-prone. We found that this situation could be produced in cancer cells by reducing the mevalonate pool to a stage that cholesterol synthesis was extensively inhibited A and cell growth was marginally arrested. In other words, the lovastatin-treatment (to the HCC-like Hep 3B cells) sensitized the residual cancer cells to a second anticancer drug or an apoptotic agent. By bringing cancer cells to a stage between severe cholesterol synthesis inhibition and marginal growth arrest by lovastatin or other liver-specific statins (such as lipitor), the use of statins and other anticancer agents (examples of in vitro studies: Agarwal, B. et. al., 1999. Clin. Canc. Res. 5: 2223-2229; Soma, M. A. et. al., 1995. 55: 597-602.), such as paclitaxel, may be expanded. It may also reduce the dose required for an effective cancer therapy.

We have successfully tested our hypothesis by a combination treatment of lovastatin with paclitaxel (For review on paclitaxel see example ref. Fan, W. 1999. Biochem. Pharmacol., 57: 1215-1221; Guchelaar, H. J., et. al., 1994. Clin. Oncol., 6: 40-48.) which is an anticancer agent which is not effective in the treatment of human HCC. Combination of lovastatin and antioxidants as apoptotic agents has also been tested. We used a combination of lovastatin and extracts from Ganoderma (Won, S. J., et. al., 1992. Jpn. J. Pharmacol., 59(2): 171-176; Furusawa, E., et. al., 1992, Phytother. Res., 6: 300-304; Lee, S. S., et. al., 1984. J. Chin. Oncol. Sci., 5: 22-28; Wang, S. Y., et. al., 1997. Int. J. Cancer, 70(6): 699-705; Toth, J. O., et. al., 1983. J. Chem. Res., 299; Lee, S. S., et. al., 1995. J. Chin. Med., 6(1): 1-12; Gan, K. H., et. al., 1998. J. Natur. Prod., 61: 485-487.) and CAPE from propolis (Grunberger, D., et. al., 1988. Experientia, 44: 230-232; Chiao, C., et. al., 1995. Cancer Res., 55: 3576-3583.). Previous studies from our laboratory and others have demonstrated that Ganoderma extract is hepatoprotective and causes apoptosis in hepatoma cells (including Hep 3B). Propolis, the extract of honey-bee hive, contains caffeic acid phenethyl ester (CAPE) as a mild antioxidant and apoptotic agent to the transformed cells. We have extensively examined the potential of these agents as the second apoptotic agents to the lovastatin-primed Hep 3B cells.

The cytostatic/cytotoxic and apoptotic activities in vitro of the combination treatment of lovastatin (G0/G1 arrest) with paclitaxel (G2/M arrest), apoptotic antioxidants (CAPE; G1 arrest), or Chinese medicinal fungus Ganoderma (a fraction called GL-M1, see below; G2/M arrest) were significantly enhanced as compared with those treated without lovastatin (p=0.014). For agents causing growth arrest at different cell cycle stages, sequential treatment was surprisingly better to suppress cancer cell growth. For example, first treatment with lovastatin followed by treatment with paclitaxel (at a lower dose than that in individual treatment) to Hep 3B cells was found very effective (comparing Table 12-13 to Table 10). Ganoderma alone is effective at high concentration to reduce the viability of Hep 3b cells (Table 17). Using lovastatin and Ganoderma as combination treatment was very effective in reducing the viability of Hep 3B cell but damaged HepG2 cells even more (Table 21). But, surprisingly and satisfactorily, treatment with lovastatin first then followed by Ganoderma alone very effectively diminished the viability of Hep 3 cells and leave HepG2 cells relatively unharmed. (Table 22). These results suggested that a successful method in treating cancer with one agent (such as lovastatin) to sensitize the cancer cells and by another agent (such as paclitaxel or Ganoderma) that arrest cancer cell growth at a stage later than the first one dose, would be of sequential administration of the first one followed by the other. The method may require carefully fine tune the timing and dosage of each agent and according to the nature of different cancers and agents.

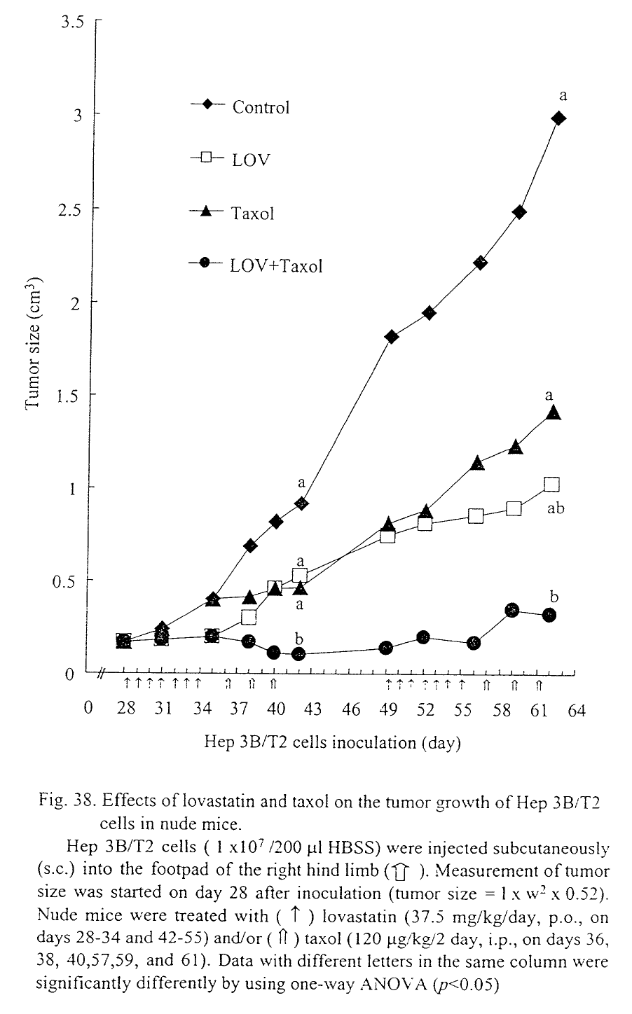

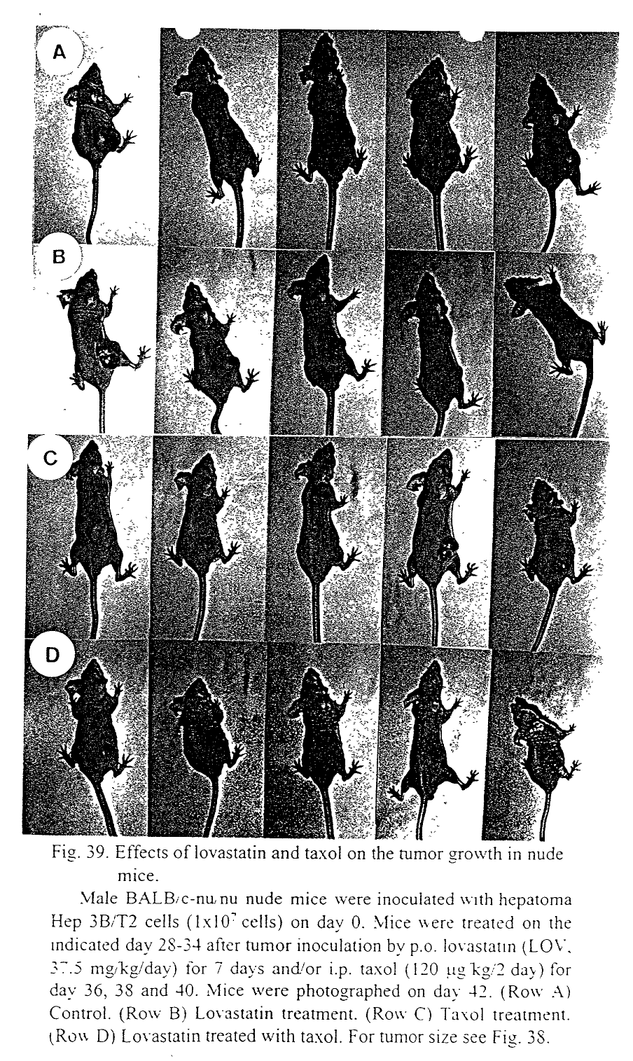

Results based on the above consideration from animal study (BALC/c-nu/nu mice inoculated with Hep 3B/T2 cells) arc very promising. Nude mice bearing human liver cancer tumor were fed with lovastatin for 7 days and subsequently injected with paclitaxel for 3 days. The descriptive statistics were summarized as mean±STD. For statistical analysis, Student's t-test was used to determine significance of mean differences between groups. As shown in FIG. 38, after two cycle of such treatment, the tumor size of the sequentially treated group as well as the paclitaxel-alone group) was significantly reduced (0.32+0.46 cm³ and 1.43±0.46 cm³, respectively) as compared with the control group (2.50±1.85 cm³) at p=0.05 level. The lovastatin-alone group was reduced to 1.26±1.82 cm³ although it was not significantly different from the control group.

We also have successfully tested our hypothesis in vivo by a sequential treatment of lovastatin followed by Ganoderma. A partially purified fraction from Ganoderma (called fraction GL-M1-C-M-14) with significant apoptotic effect determined in vitro was used for animal study to elucidate the pharmacological potential by using Hep 3B/T2 hepatoma cell-implanted nude mice as the animal model.

Animal study results (FIG. 41) demonstrated that the tumor sizes of control (Ct, n=6), lovastatin (L, n=6), GL-M1-C-M-14 (C14, n=6), and sequential treatment of lovastatin and GL-M1-C-M-14 (LC14, n=6) groups at the end of one cycle of treatment were 3.04±1.61 cm³, 2.24±2.38 cm³,1.28±1.03 cm³ and 0.91±0.71 cm³, respectively. The reduction of tumor size or control of tumor growth by combination treatment in a sequential manner was significantly more effective (p=0.014), but treatment with Ganoderma extracts alone also showed significantly reduced tumor size compared to the untreated (p=0.048). In conclusion, this study further demonstrated that growth-arrested human hepatoma cells (Hep 3B) became more sensitive to apoptotic agents, if the cellular mevalonate pool was significantly reduced. The Chinese medicinal fungus Ganoderma contained apoptotic natural products, which are oxygenated triterpenes, promoted hepatoma cell apoptosis in vitro and in vivo. This study suggests novel combination chemotherapy of human HCC. It also illustrates the potential application of Chinese medicinal fungus Ganoderma as the adjuvant therapy for human HCC.

GL-M1 and GL-M1-C-M-14 Ganoderma extraction fractions were prepared as following: Dried Ganoderma fruiting bodies were grinded first, then extracted with 10 time (weight ratio) ethaol overnight at room temperature. After filtering, the extract was concentrated by low pressure evaporation to obtain GL-M1. GL-M1 was adsorbed onto silica gel. The silica gel was then packed into a column and eluted sequentially with five solvents. They are hexane:ethyl acetate (1:1 v/v), hexane:ethyl acetate (1:2 v/v), ethyl acetate, ethyl acetate:methanol (1:1 v/v), and finally methanol. Fractions collected from elution were assayed for their anti-tumor cell activities. High activity was recovered from the early methanol elution and was collected as fraction GL-M1-C-M-14.

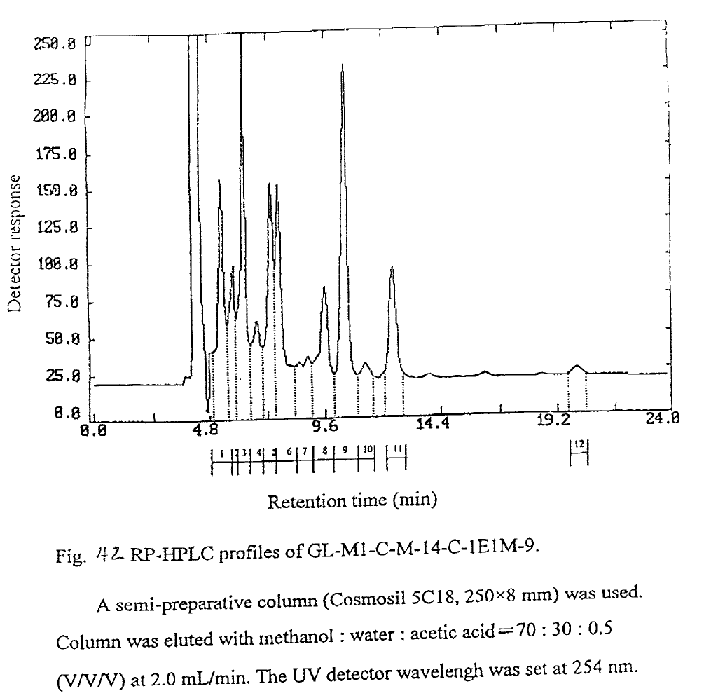

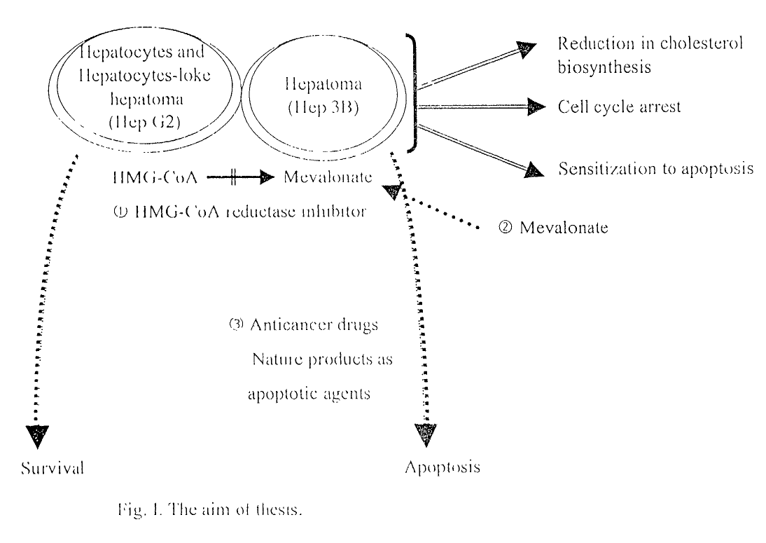

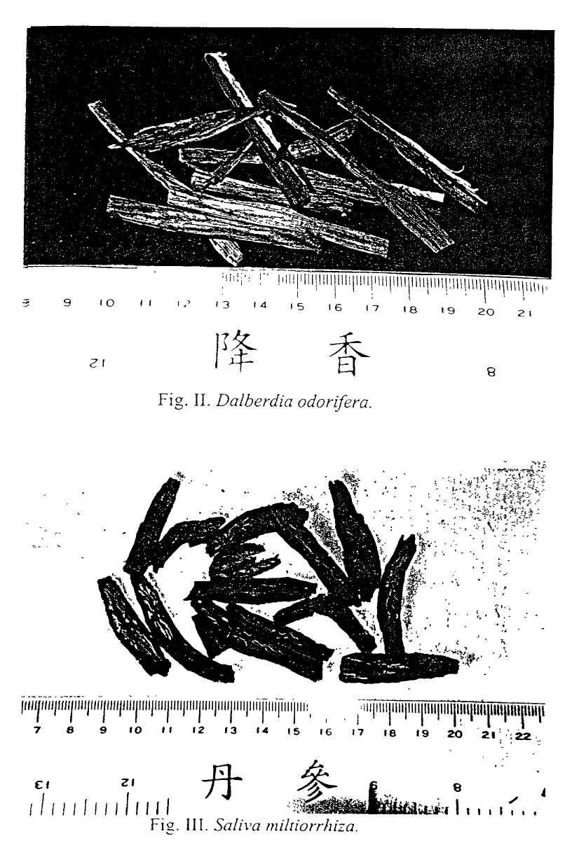

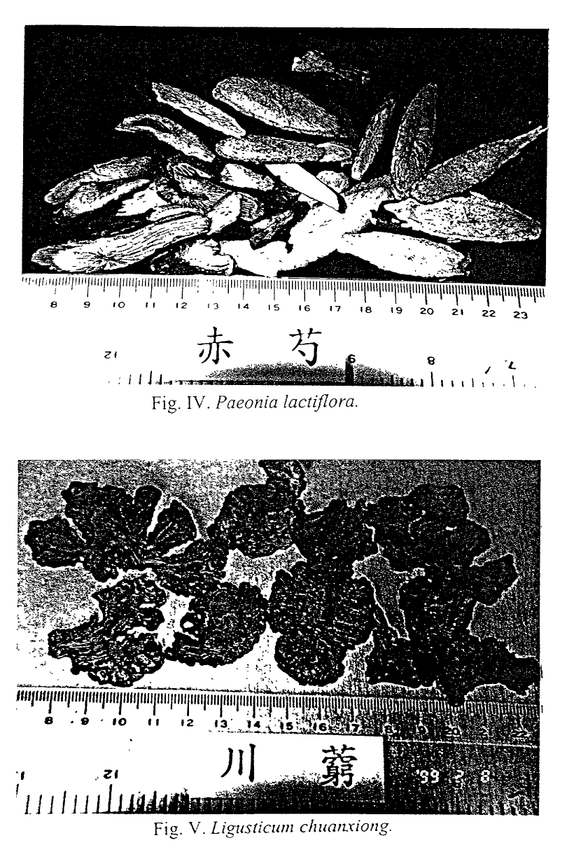

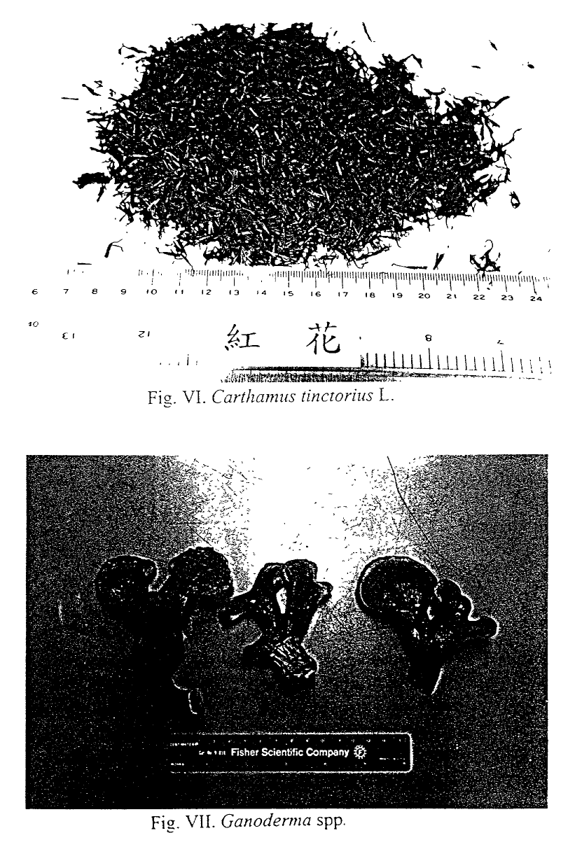

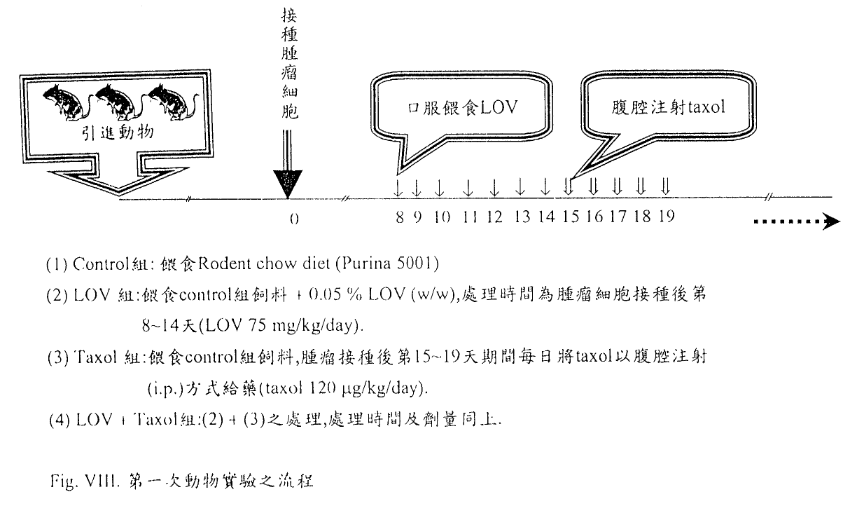

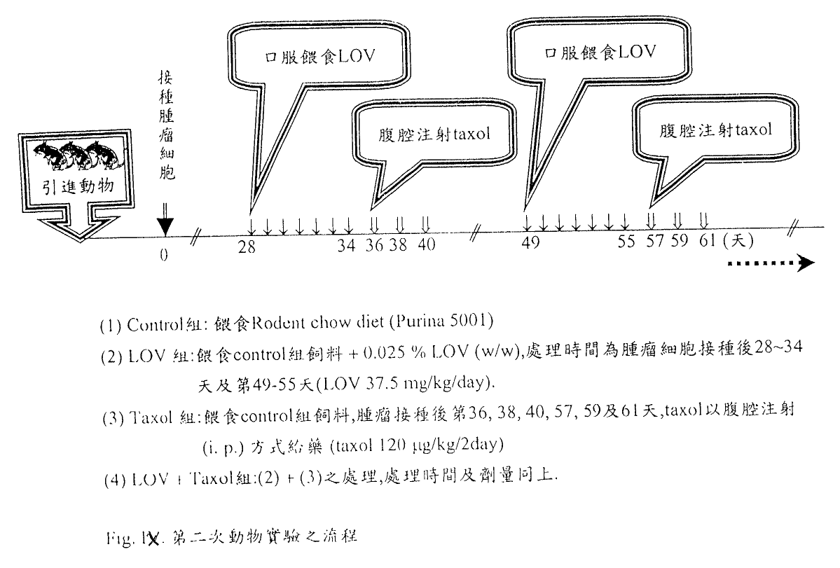

GL-M1-C-M-14 was further fractionated according to the following steps: GL M1-C-M-14 was applied to a silica gel column. The column was then washed sequentially with five solvent systems which were ethyl acetate, ethyl acetate:methanol (1:1 v/v), ethyl acetate:methanol (1:2 v/v), ethyl acetate:methanol (1:3 v/v), and methanol. Bioassay indicated that the fraction eluted with ethyl acetate:methanol (1:1 v/v) contained the highest concentration of active material. This fraction was named GL-M1-C-M-14-C-1E1M-9. Reverse phase HPLC analysis of GL-M1-C-M-14-C-1E1M-9 on Cosmosil 5C18, 250×8 mm, eluted with methanol:water:acetic acid (70:30:0.5 v/v/v) at 2.0 ml/min showed that GL-M1C-M-14-C-1E1M-9 contains at least ten major compounds and were eluted between 4 min and 22 min with majority eluted between 4.5 min and 13 min (See FIG. 42). These compounds are mostly oxygenated triterpenes. There are more than one hundred triterpenes that have been identified in the literatures (for review, see Shiao, M. S. et al., in Amer. Chem. Soci. Symp. Ser. V. 547.“Food phytochemistry for Cancer prevention II”; Editors: C. T. Ho et al., Chapter 35.). There may be one or more triterpenes that are anti-cancer. One or more of the major active triterpenes are likely to be contained in GL-M1-C-M-14-C-1E1M-9.

A nutraceutical composition which contains the above described anti-cancer oxygenated triterpenes can be used alone or in a sequential manner help by another agent (to presensitize the cancer cells), to treat cancers, including liver cancer. Such a nutraceutical composition can be part of a drink, a food, a dietary supplement or a botanical drug. The nutraceutical composition can be of a crude extract of Ganoderma fruiting bodies. Or the crude extract can be further purified to enrich the said oxygenated triterpenes by various procedures including but not limited to solvent partitioning or liquid chromatographic techniques which are known in the field of chemistry.

In summary, we have demonstrated that the coordination of multiple mevalonate-demanding routes is disturbed in several types of cancer, particularly in HCC. The HCC-like Hep 3B cells, which were used as an in vitro model in our study, were more mevalonate demanding. Reduction of mevalonate pool, to a stage of profound suppression in cholesterol synthesis and marginal cell growth arrest enhances the sensitivity and selectivity of the hepatoma cells to another anticancer drugs, like paclitaxel, antioxidants as apoptotic agents, or the Chinese medicinal fungus Ganoderma,. Animal study further supports these observations. This hypothesis and the potential beneficial effects on human HCC can also be verified by using health foods or dietary supplements. A combination of monacolin K (lovastatin)-containing products and Ganoderma or CAPE-containing propolis may reach the same goal if the pharmacological potential of each active ingredient is carefully taken into consideration. Our study opens a new way of therapy for the treatment of human hepatocellular carcinoma and other cancers.

The working hypothesis is based on the essential role of mevalonate to cell function. It is almost impossible for the proliferating cancer cells to produce lovastatin (and other statins) resistance to avoid sensitization to the following anticancer agents. The second anticancer agents can have many choices. It can be a known anticancer agent of new application (such as paclitaxel) or a new anticancer agent used in lower dose. A cocktail treatment is also possible.

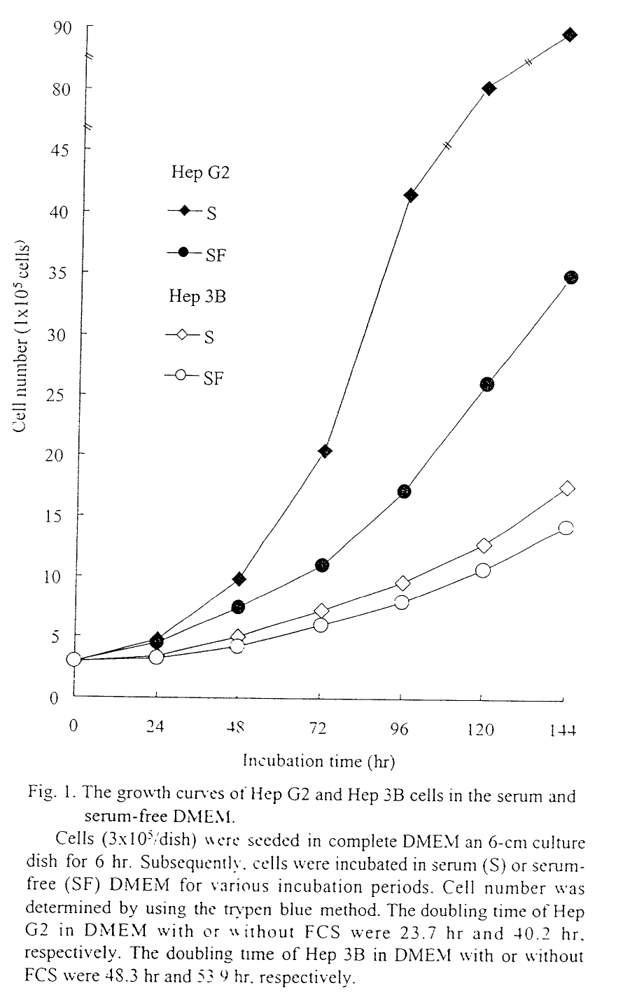

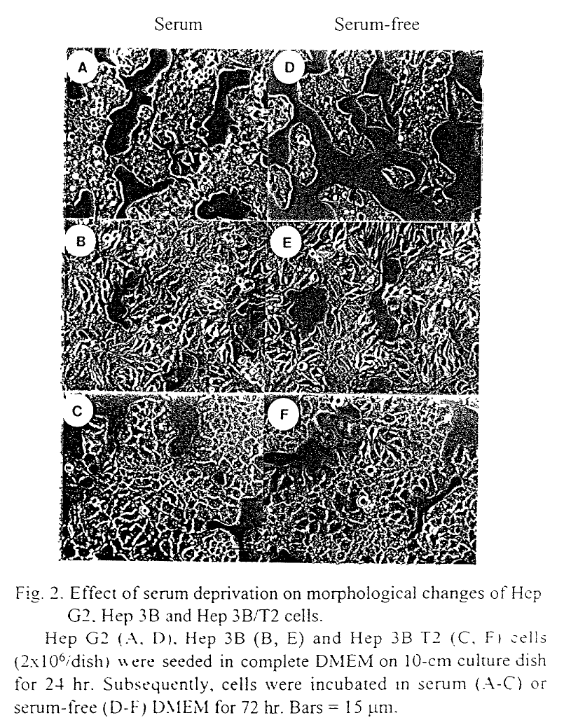

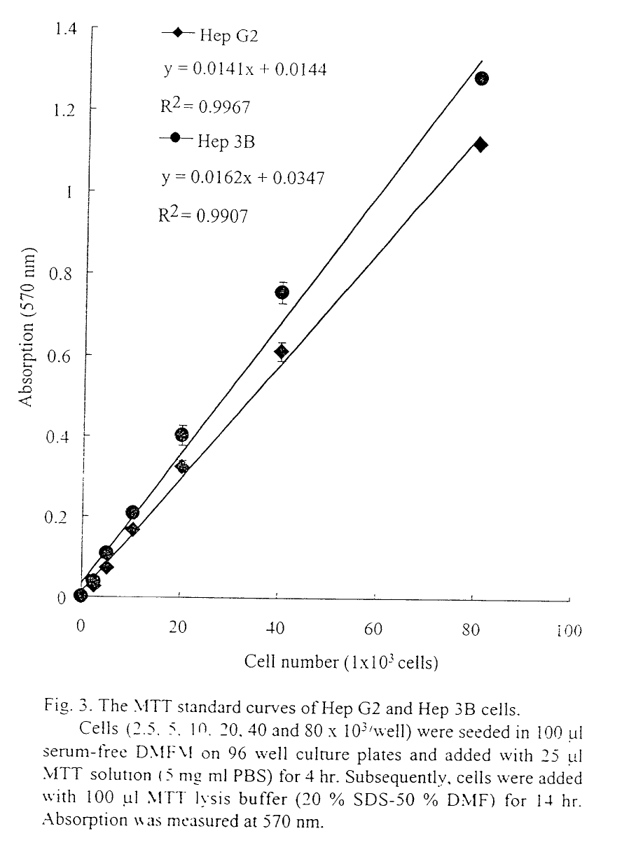

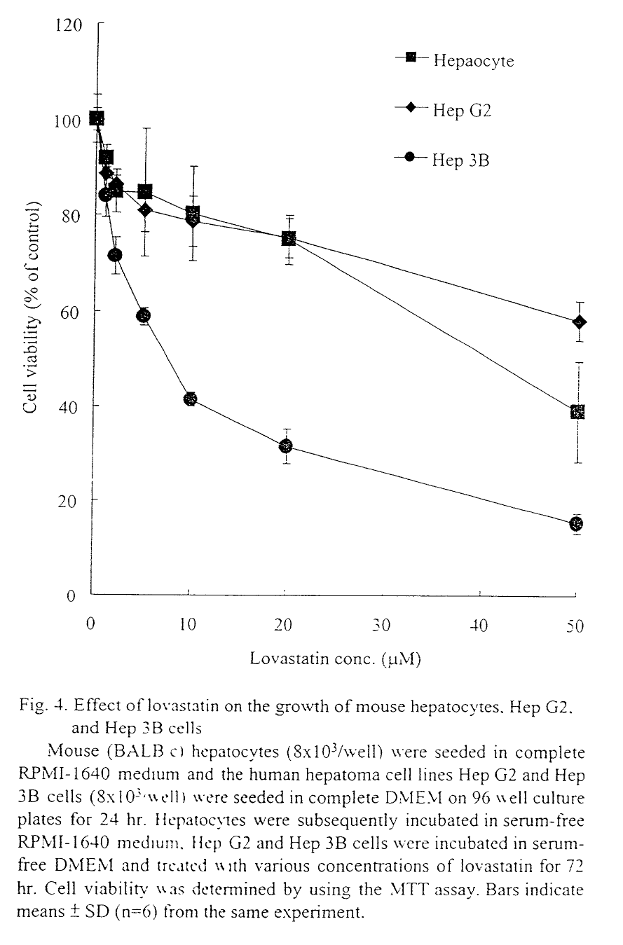

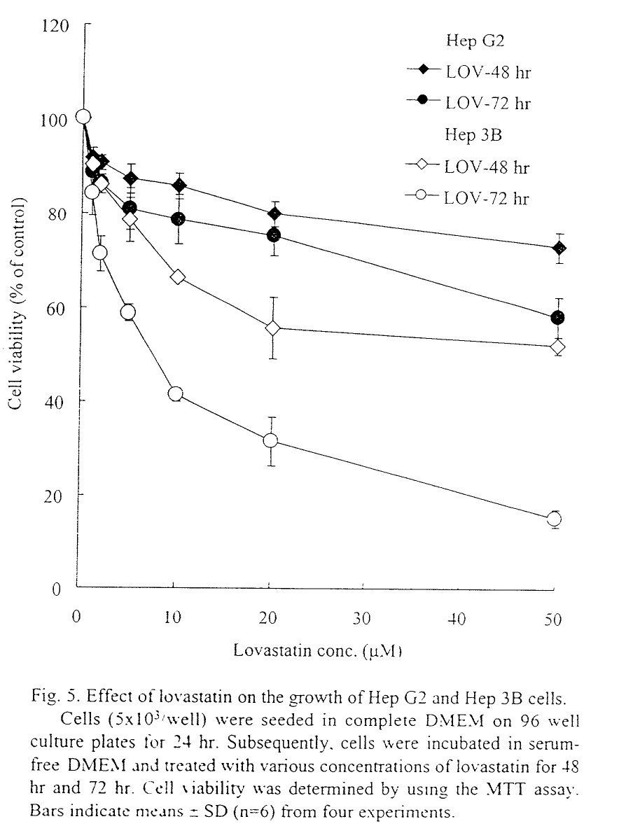

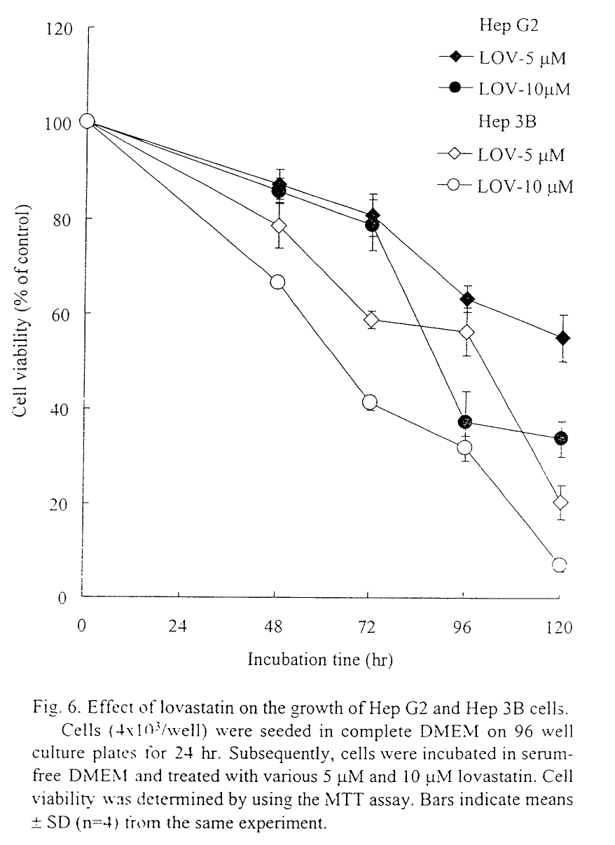

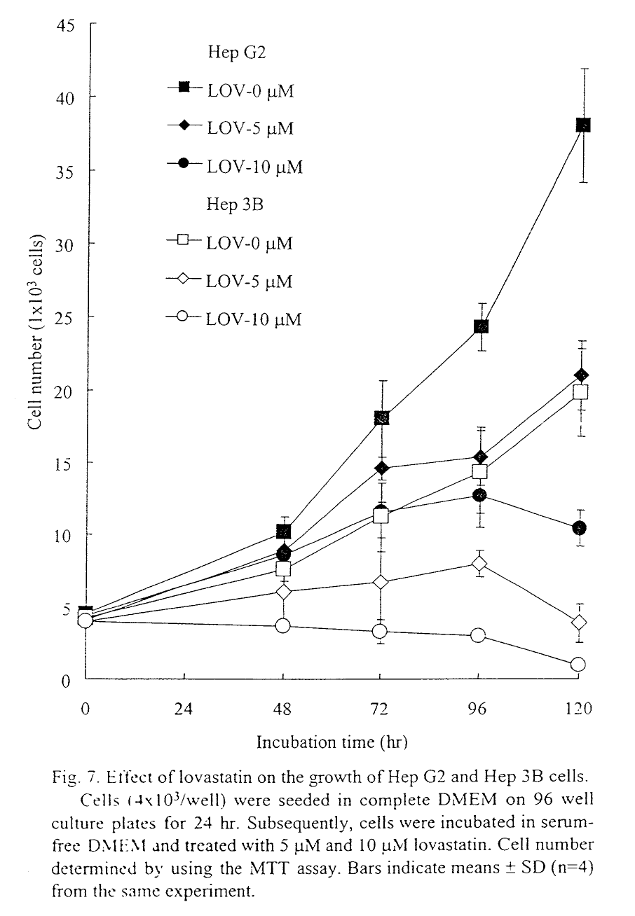

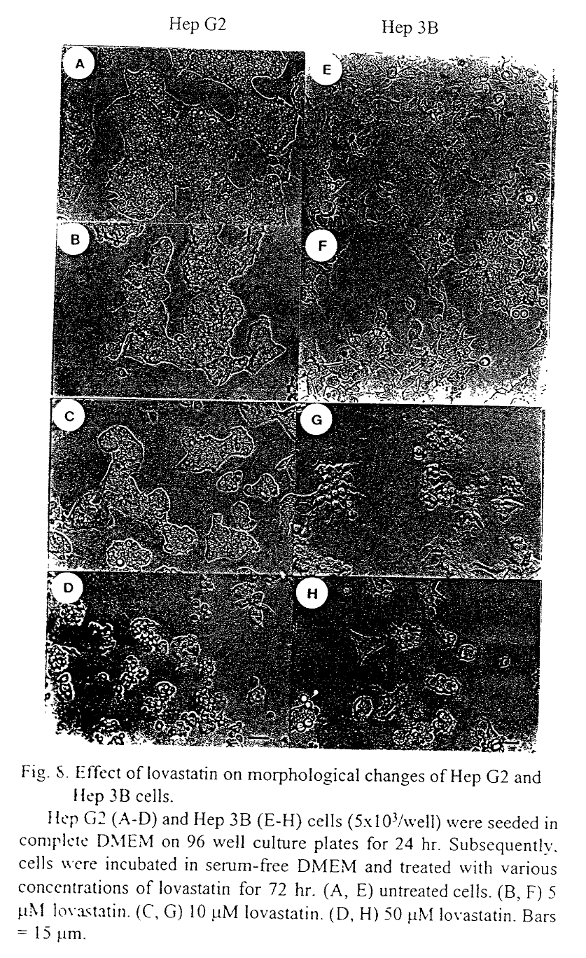

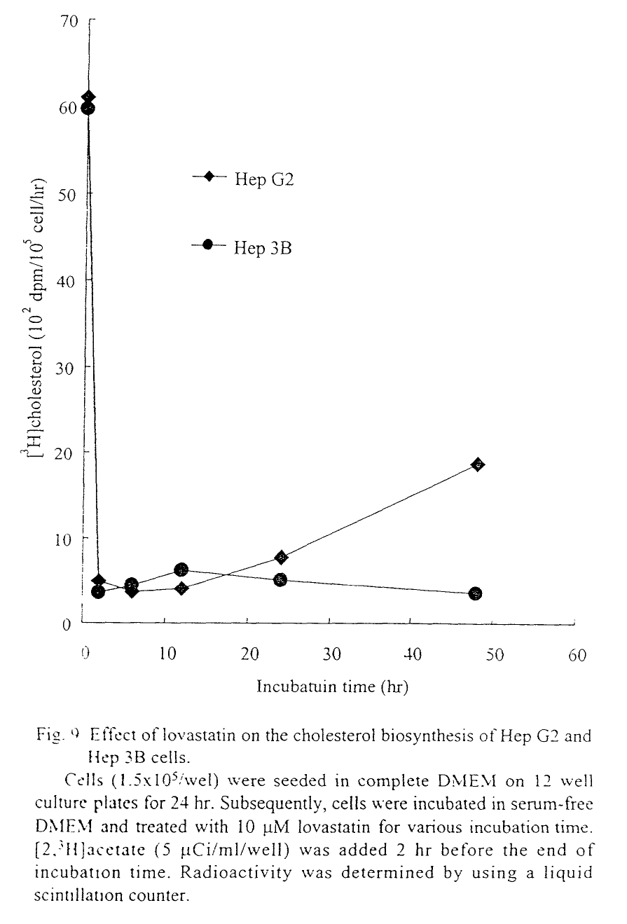

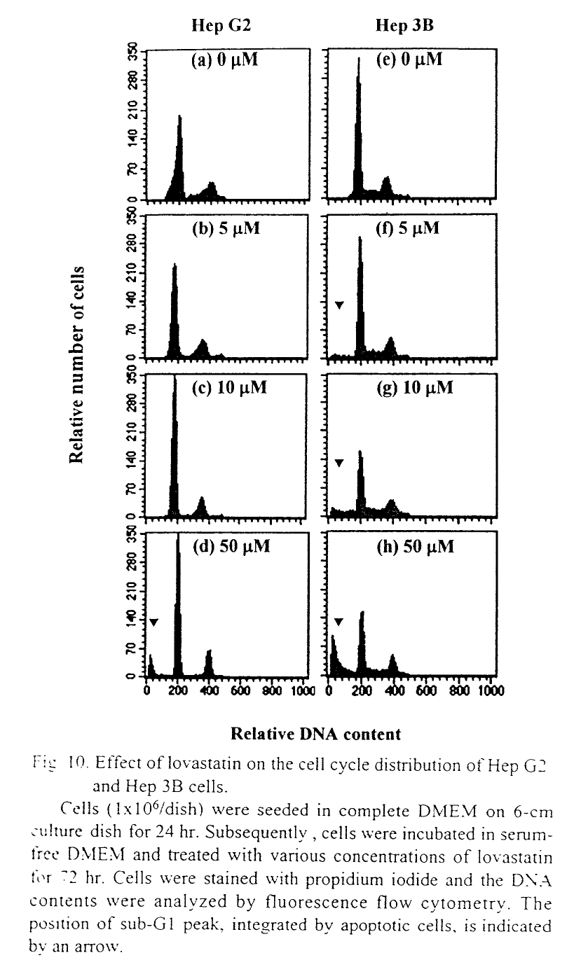

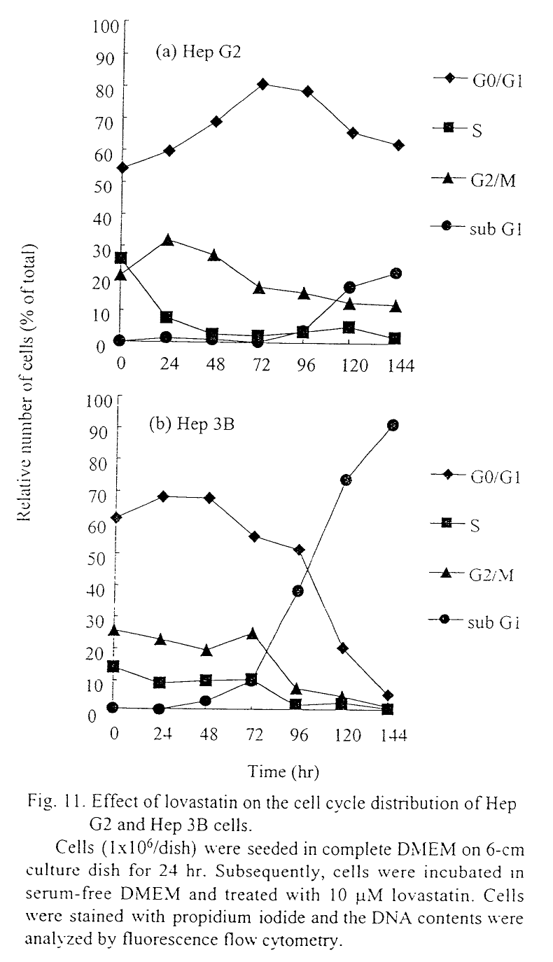

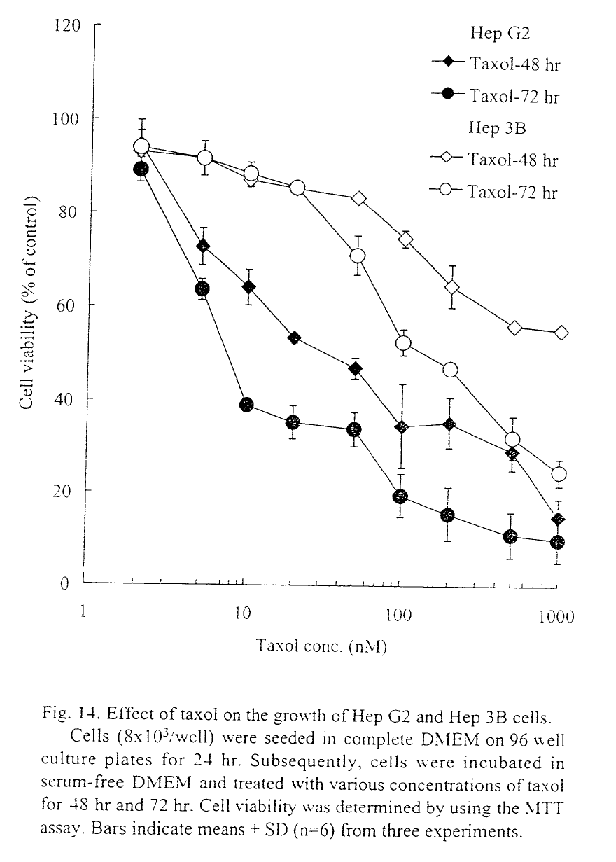

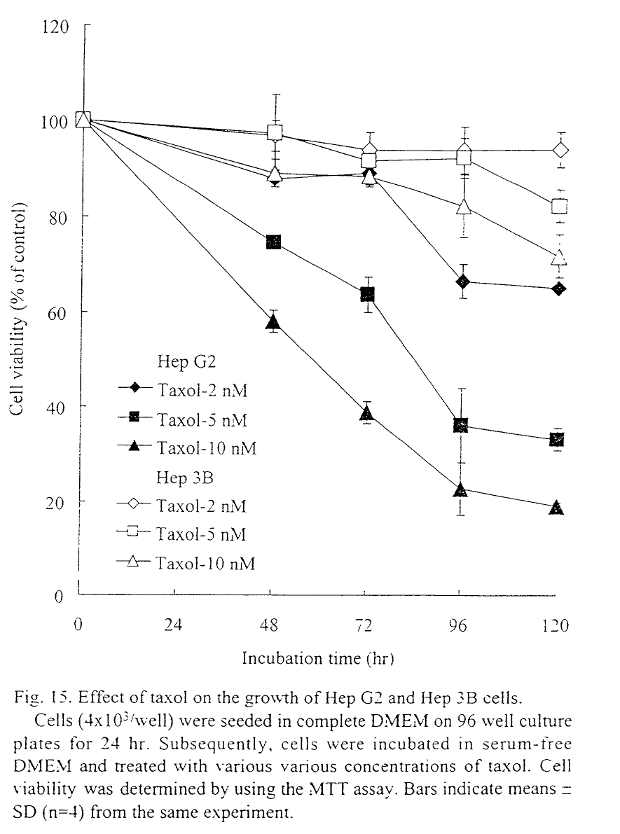

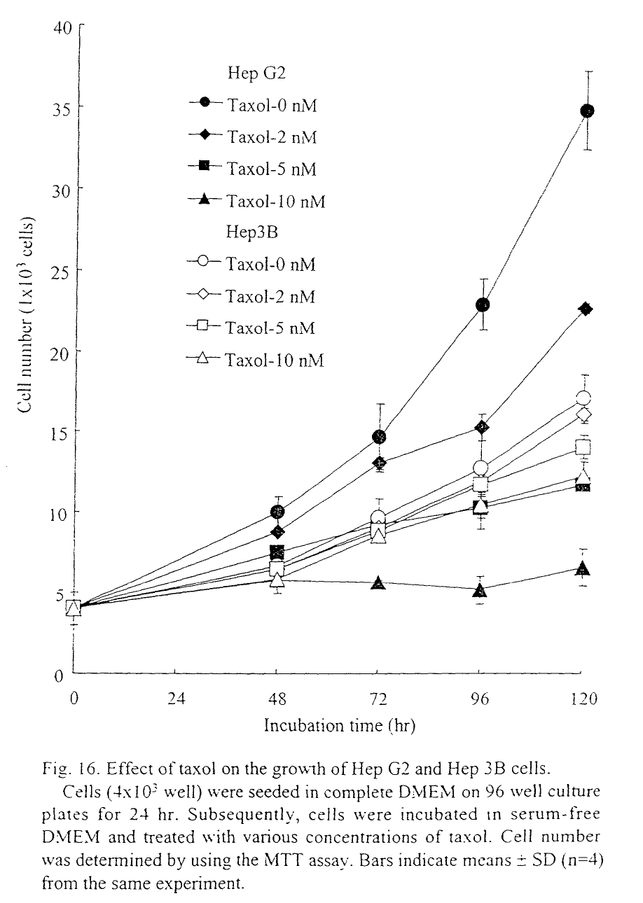

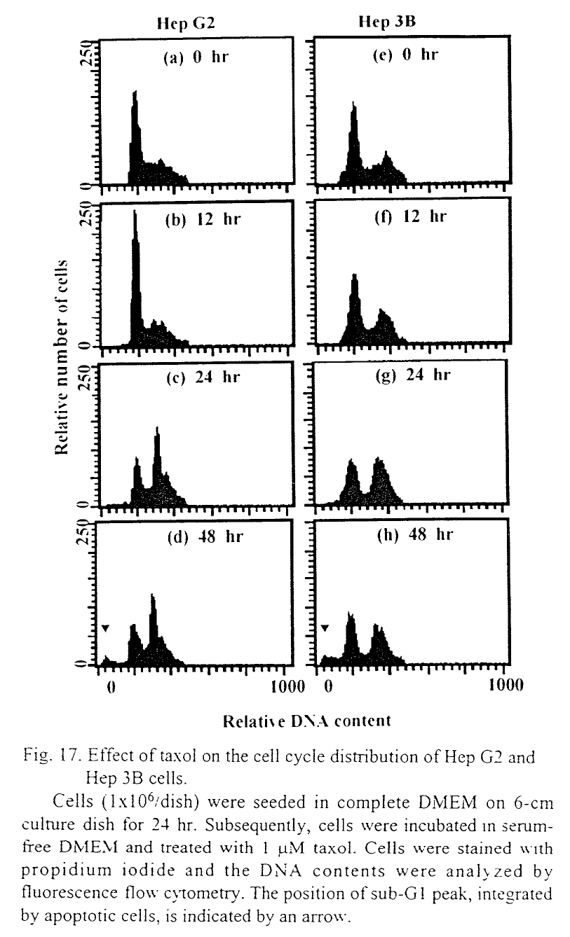

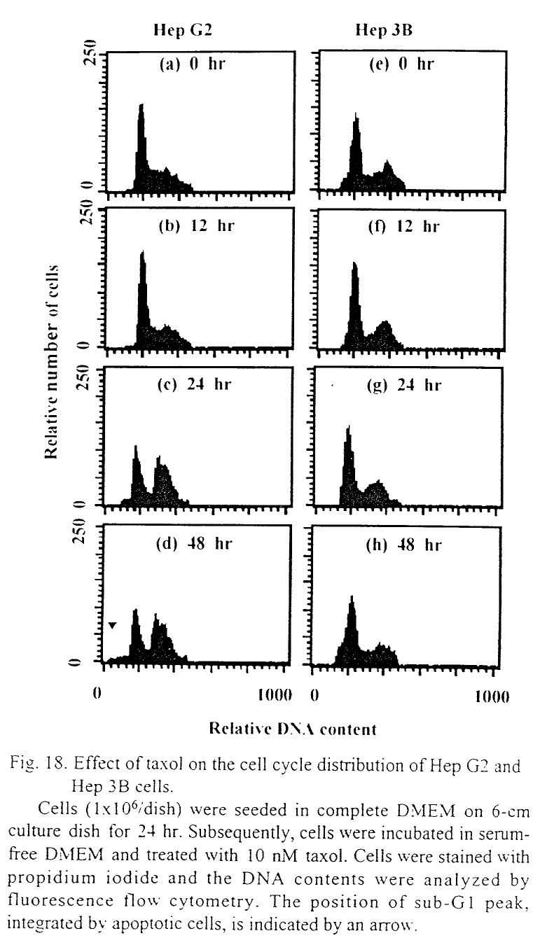

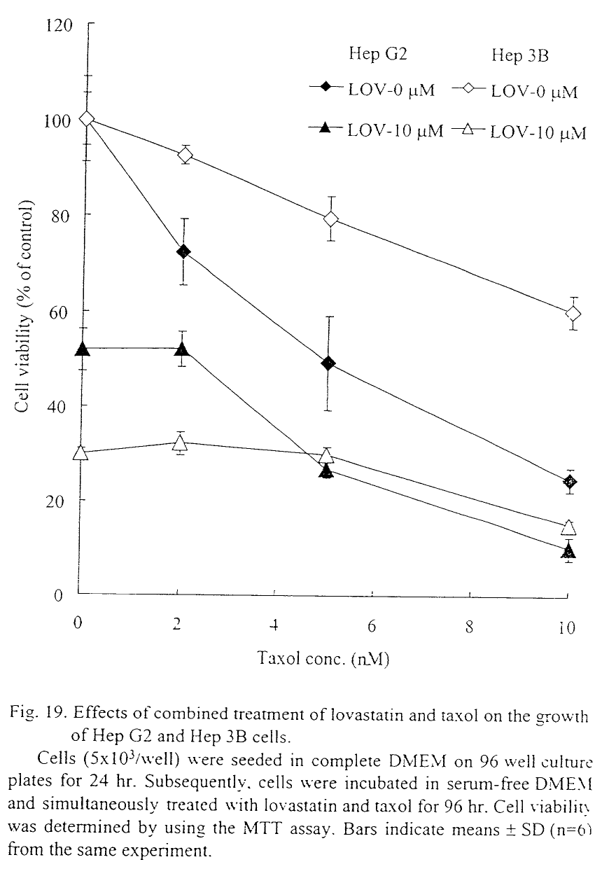

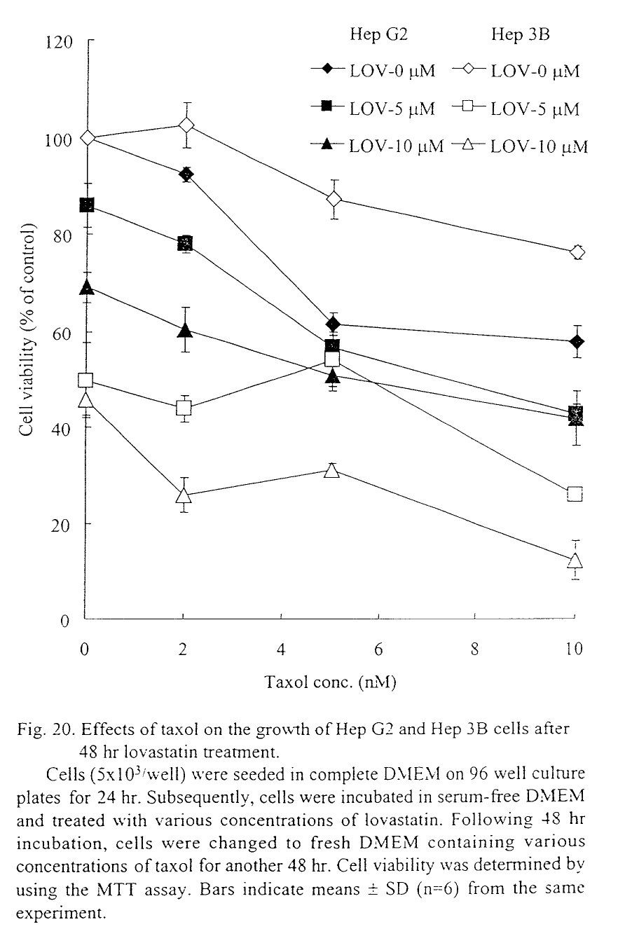

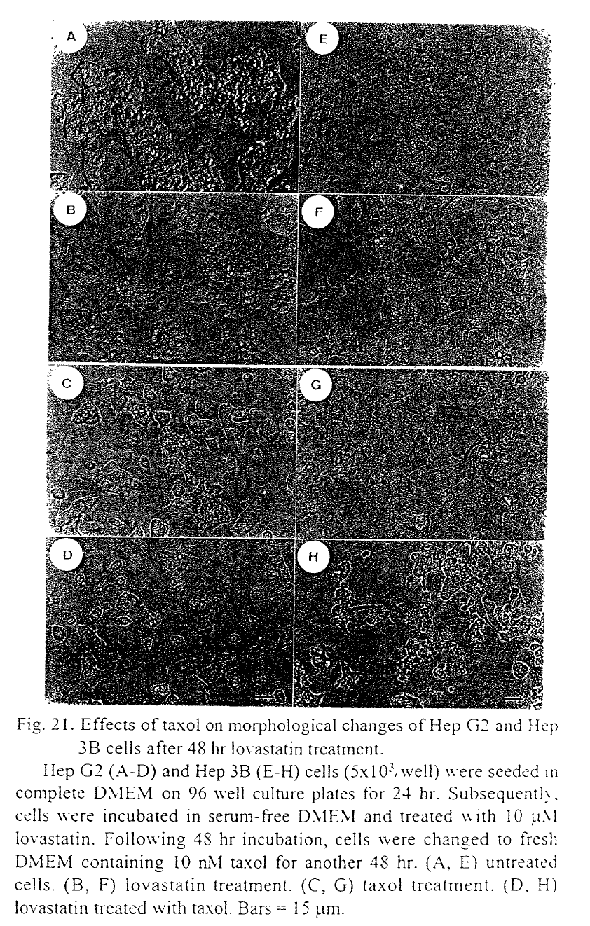

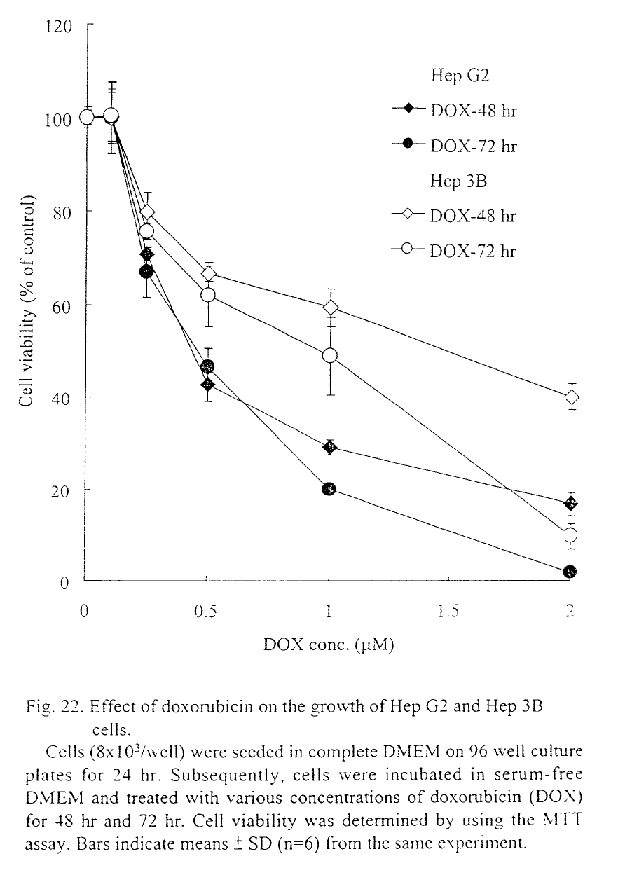

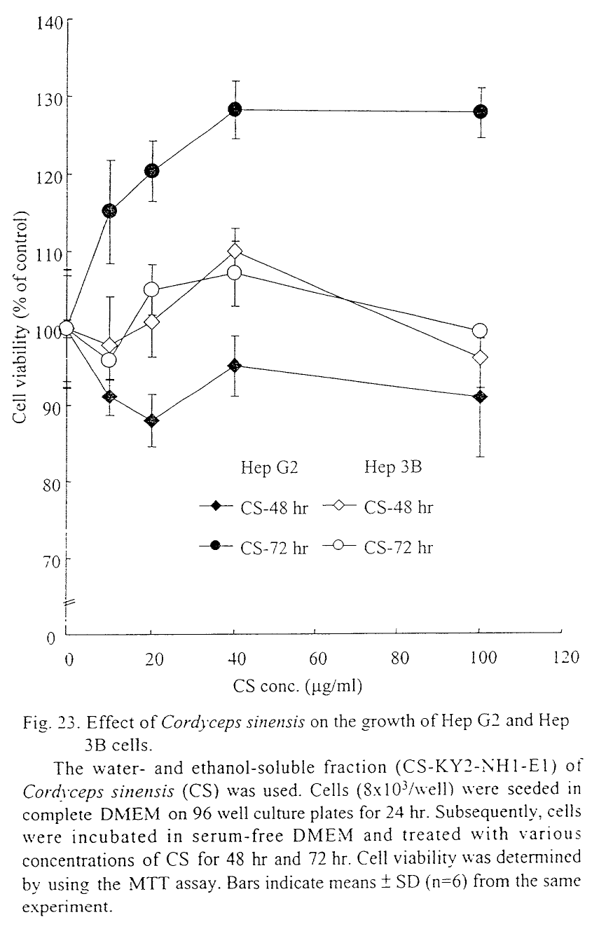

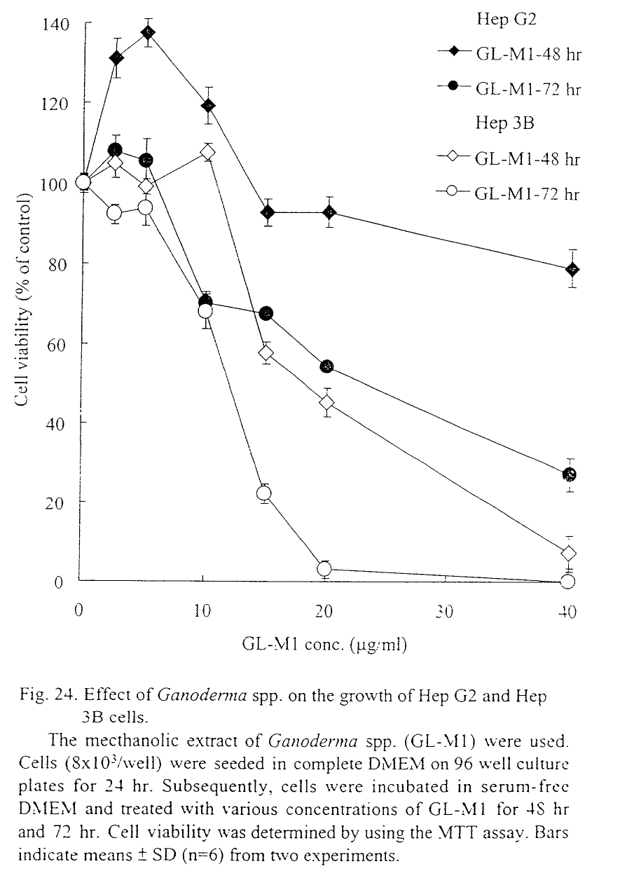

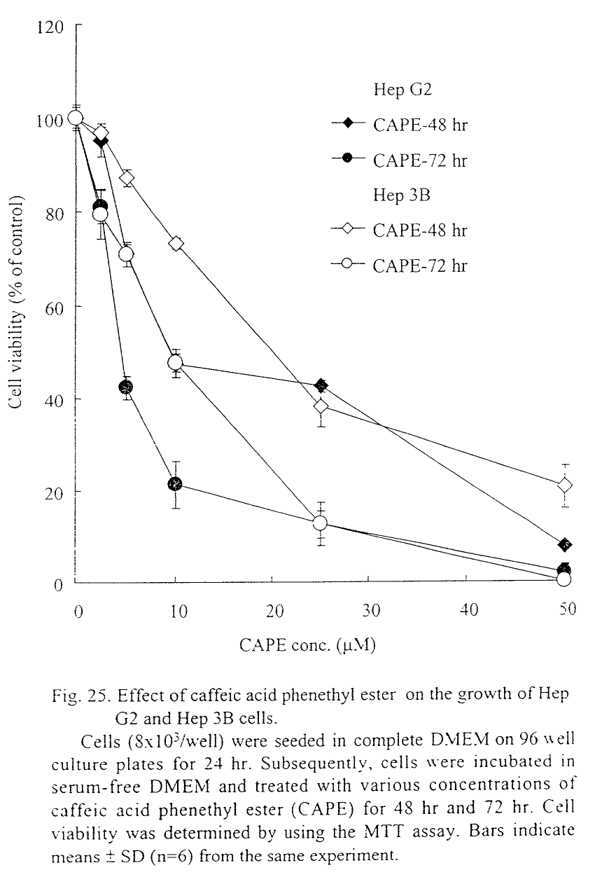

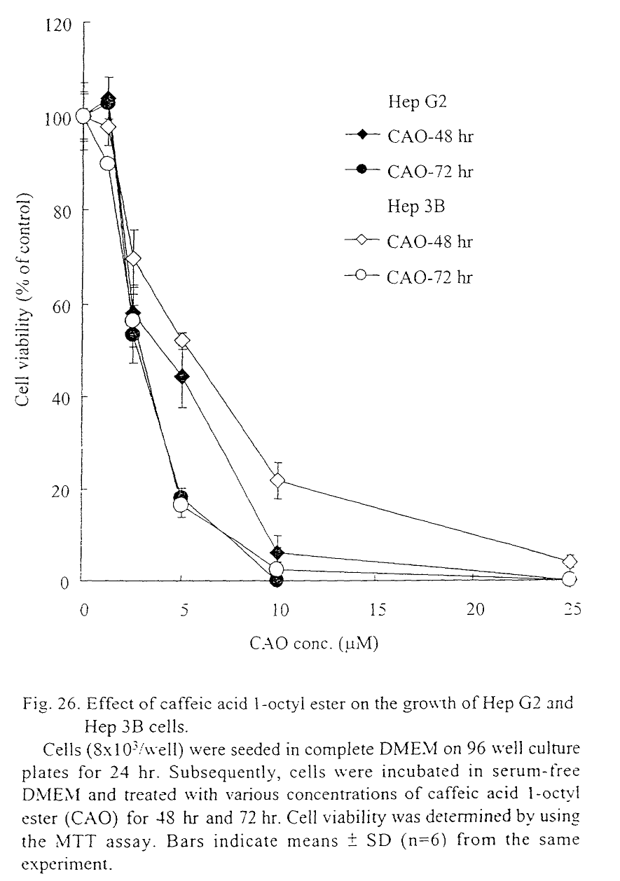

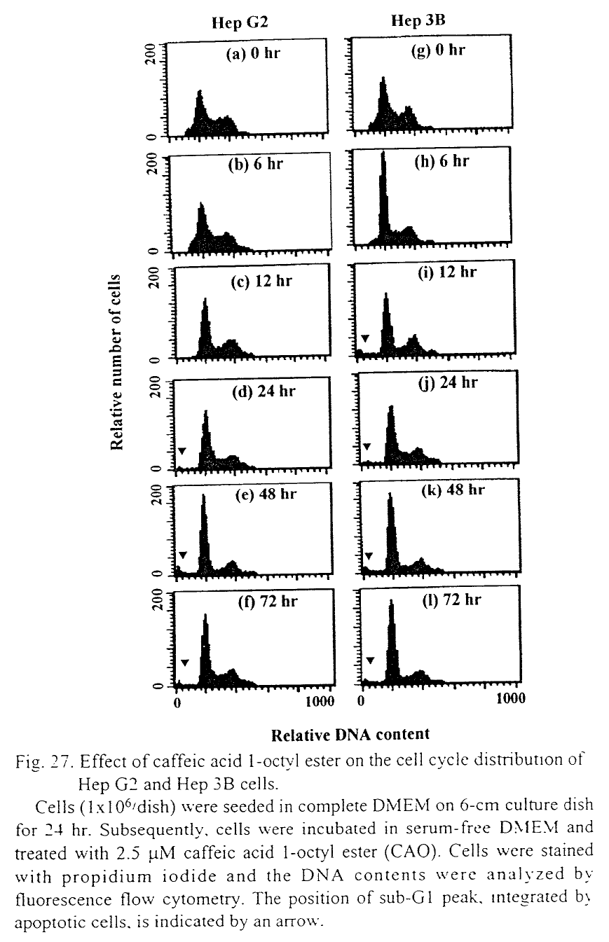

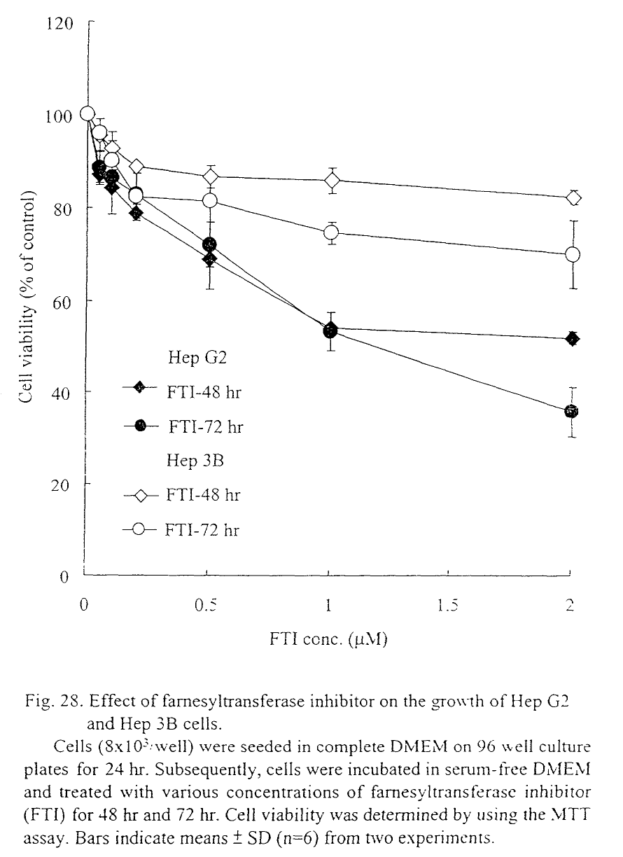

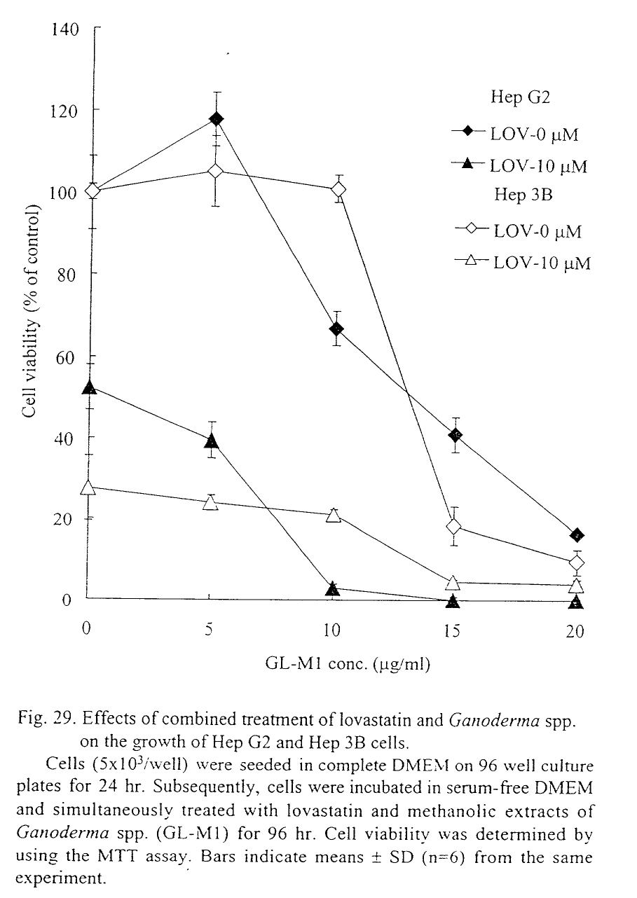

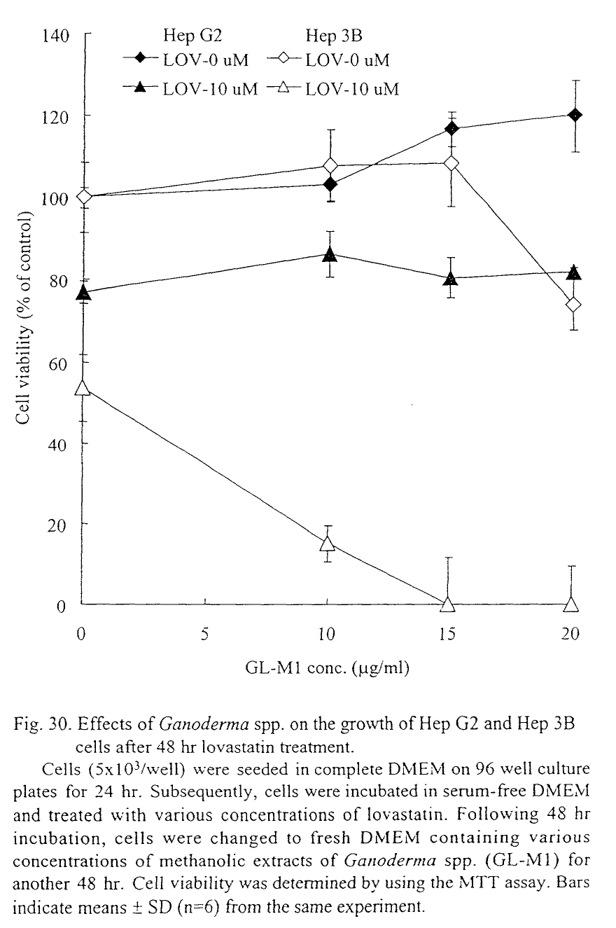

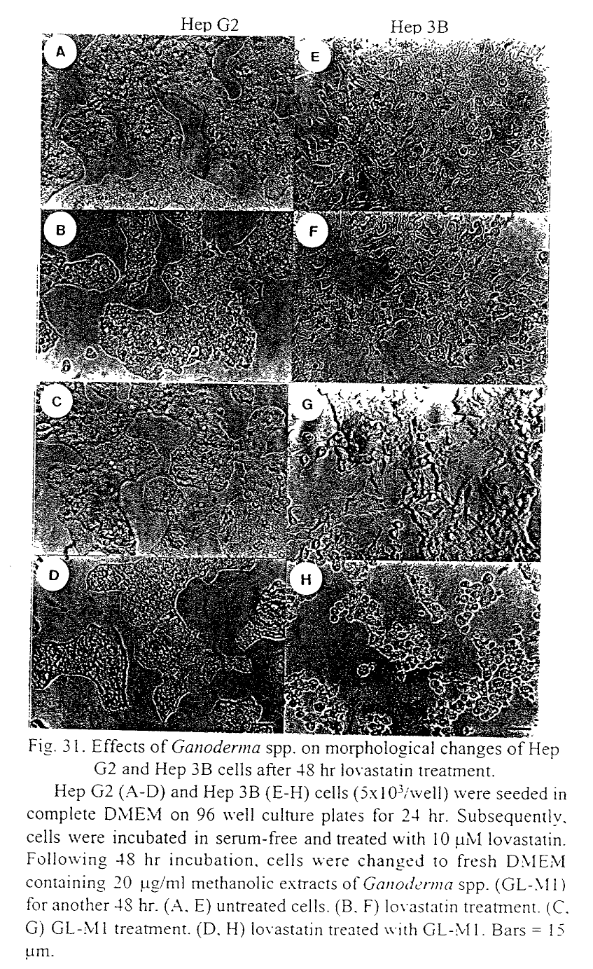

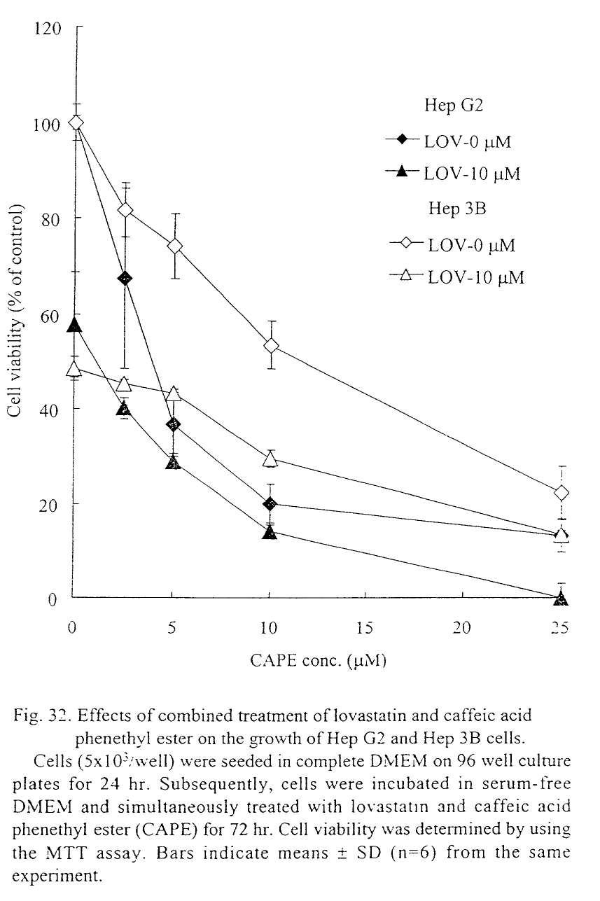

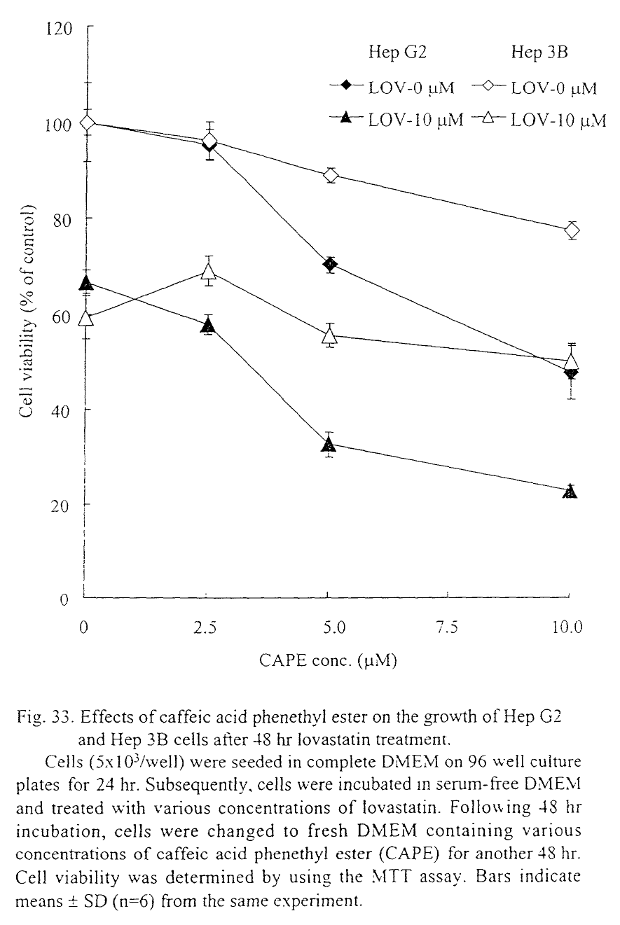

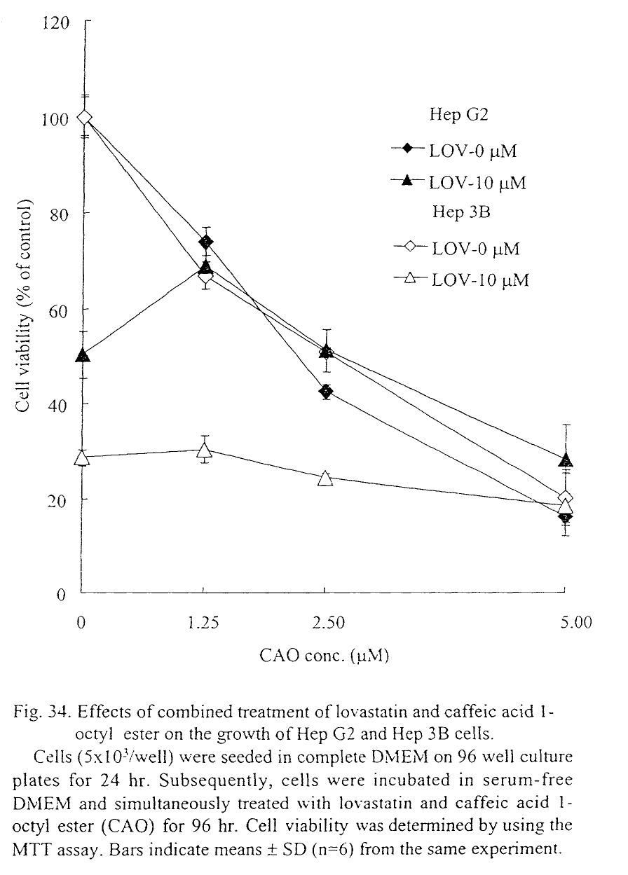

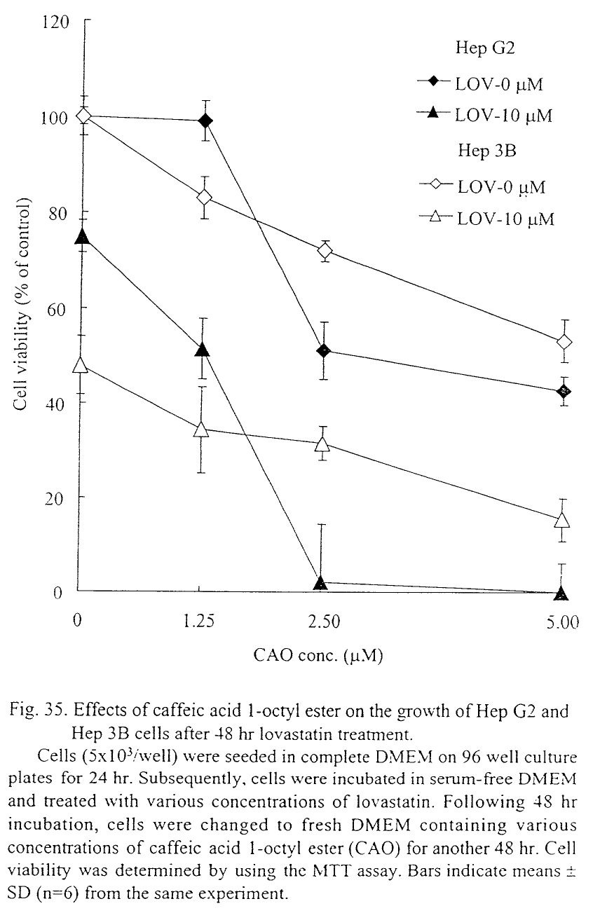

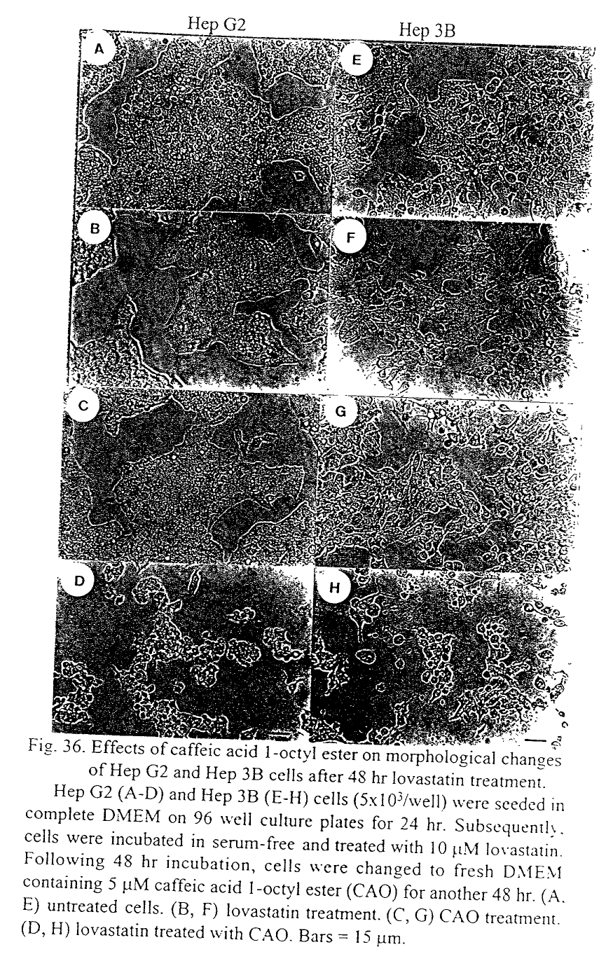

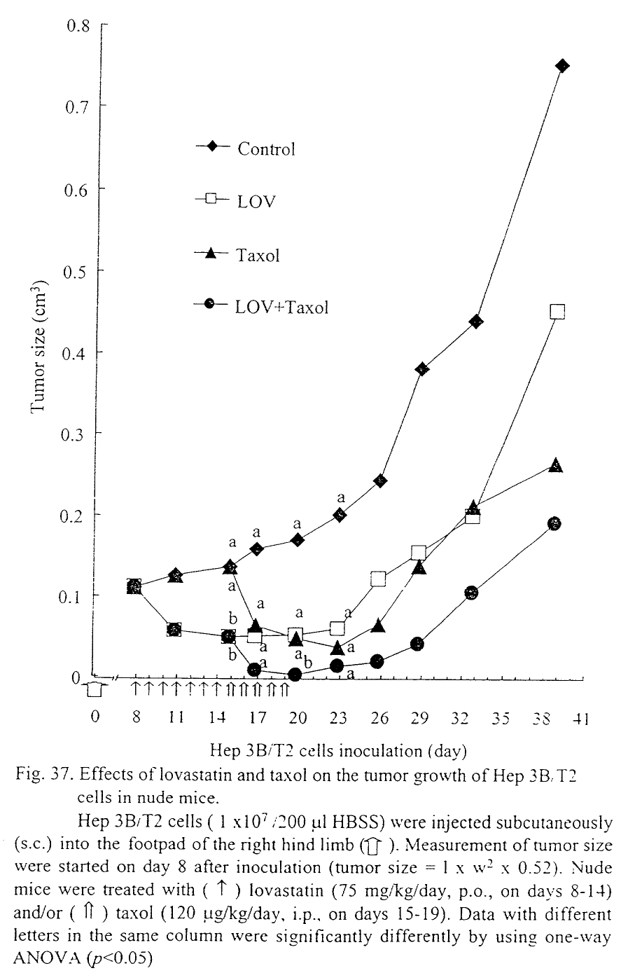

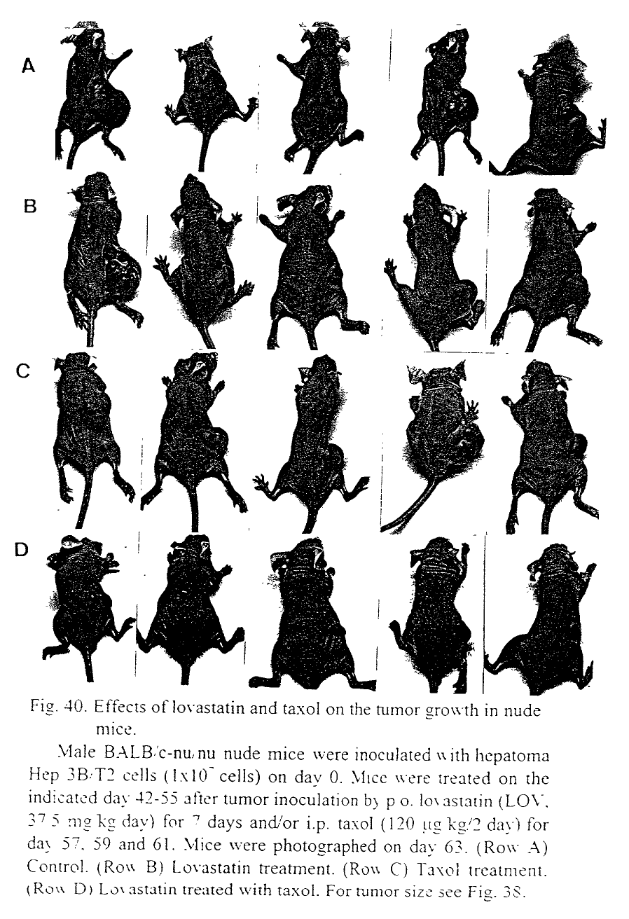

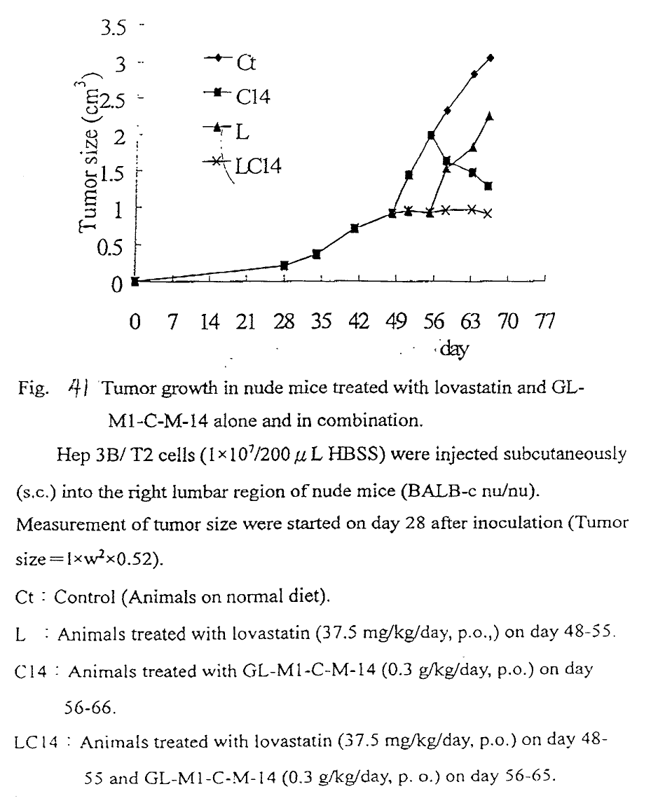

The Tables 1-29 and FIGS. 1-42 and I-IV in the following pages (Attachment I) describe some specific embodiments of the present invention. The abbreviations are defined in Attachment II. Part of the data in this application was mentioned in a thesis abstract titled “Studies on the Coordination of the Multiple Routes of Mevalonate-Dependent Pathways and Selective Interruption in Human Hepatoma Cell Lines Hep G2 and Hep 3B”. The abstract is indexed under “NATIONAL DOCTOR and MASTER THESIS ABSTRACT SEARCH SYSTEM” with a serial number 87YM000603003 and dated Jul. 31, 1999. The thesis abstract is also indexed under “YANG MING UNIVERSITY LIBRARY NETWORK” with a file number 1999003. Full text of the thesis is not available to the public.

TABLE 1
Effect of serum on the growth of Hep G2 and Hep 3B cells.

| | Cell number | | | |
|---|---|---|---|---|
| Incubation | (1 × 10⁵ cells) | | | |
| time | Hep G2 | | Hep 3B | |
| (hr) | Serum | Serum-free | Serum | Serum-free |
| 0 | 3.0 | 3.0 | 3.0 | 3.0 |
| 24 | 4.8 | 4.5 | 3.4 | 3.2 |
| 48 | 9.8 | 7.5 | 5.0 | 4.2 |
| 72 | 20.4 | 11.0 | 7.3 | 6.0 |
| 96 | 41.5 | 17.2 | 9.6 | 8.0 |
| 120 | 80.6 | 26.1 | 12.8 | 10.7 |
| 144 | 90.5 | 34.9 | 17.6 | 14.3 |
| D.T. | 23.7 | 40.2 | 48.3 | 53.9 |

1. Cells (3 × 10⁵ dish) were seeded in complete DMEM on 6-cm culture dish for 6 hr. They were subsequently incubated in serum and serum-free DMEM for various incubation times. Cell number was determined by using the trypan blue assay. Each data represents mean (n = 2) from the same experiment.
2. D. T., doubling time.

TABLE 2
Effect of lovastatin on the growth of mouse hepatocytes, Hep G2, and
Hep 3B cells.

| | |
|---|---|
| | Cell viability |
| Lovastatin | (% of control) |

| (μM) | Hepatocytes | Hep G2 | Hep 3B |
|---|---|---|---|
| 0 | 100.0 ± 5.0 | 100.0 ± 2.3 | 100.0 ± 0.9 |
| 1 | 91.9 ± 2.7^{a} | 88.7 ± 1.3^{a} | 84.1 ± 4.5^{a} |
| 2 | 85.0 ± 4.5^{a} | 86.2 ± 1.9^{a} | 71.4 ± 3.8^{b} |
| 5 | 84.7 ± 13.3^{a} | 80.9 ± 4.4^{a} | 58.8 ± 1.8^{b} |
| 10 | 80.4 ± 9.9^{a} | 78.7 ± 5.3^{a} | 41.4 ± 1.5^{b} |
| 20 | 74.9 ± 5.1^{a} | 75.3 ± 4.2^{a} | 31.5 ± 5.3^{a} |
| 50 | 39.1 ± 10.6^{a} | 58.2 ± 4.2^{b} | 15.2 ± 1.8^{c} |

1. Mouse (BALB/c) hepatocytes (8 × 10³/well) were seeded in complete RPMI-1640 medium. Human hepatoma cell lines Hep G2 and Hep 3B cells (8 × 10³/well) were seeded in complete DMEM on 96 well culture plates for 24 hr.
Hepatocytes were subsequently incubated in serum-free RPMI-1640 medium, Hep G2 and Hep 3B cells were incubated in serum-free DMEM and treated with various concentrations of lovastatin for 72 hr. Cell viability was determined by using the MTT assay. Each data represents mean ± SD (n = 6) from the same experiment.
2. Data with different superscripts in the row were significantly different by Student's t-test (p<0.05).

TABLE 3
Effect of lovastatin on the growth of Hep G2 and Hep 3B cells.

| | Cell viability (% of control) | | | | | |
|---|---|---|---|---|---|---|
| LOV | 48 hr | | | 72 hr | | |
| (μM) | Hep G2 | Hep 3B | Ratio | Hep G2 | Hep 3B | Ratio |
| 0 | 100.0^{a} | 100.0^{a} | 1.0 | 100.0^{a} | 100.0^{a} | 1.0 |
| 1 | 91.8 ± 2.1^{b} | 90.2 ± 0.3^{b} | 1.2 | 88.7 ± 1.3^{b} | 84.1 ± 4.5^{b} | 1.4* |
| 2 | 90.7 ± 1.5^{b} | 86.1 ± 2.0^{bc} | 1.5* | 86.2 ± 1.9^{bc} | 71.4 ± 3.8^{b} | 2.1* |
| 5 | 87.2 ± 3.1^{bc} | 78.6 ± 4.7^{cd} | 1.7** | 80.9 ± 4.4^{bc} | 58.8 ± 1.8^{cd} | 2.2** |
| 10 | 85.8 ± 2.7^{bc} | 66.4 ± 0.8^{de} | 2.4*** | 78.7 ± 5.3^{c} | 41.4 ± 1.5^{de} | 2.8** |
| 20 | 79.9 ± 2.7^{c} | 55.7 ± 6.6^{ef} | 2.2*** | 75.3 ± 4.2^{c} | 31.5 ± 5.3^{ef} | 2.8*** |
| 50 | 73.1 ± 3.2^{d} | 52.2 ± 1.9^{f} | 1.8** | 58.2 ± 4.2^{d} | 15.2 ± 1.8^{f} | 2.0*** |
| IC₅₀ | 97.2 ± 2.1 | 62.3 ± 7.1*** | | 63.6 ± 3.7 | 6.8 ± 1.3*** | |

1. Cells (5 × 10³/well) were seeded in complete DMEM on 96 well culture plates for 24 hr. They were subsequently incubated in serum-free DMEM and treated with various concentrations of lovastatin (LOV) for 48 hr and 72 hr. Cell viability was determined by using the MTT assay. Each data represents mean ± SD (n = 6) from four experiments.
2. IC₅₀, concentration of lovastatin required to inhibit cell viability by 50%.
3. Ratio, inhibition (%) on the growth of Hep 3B cells inhibition (%) on the growth of Hep G2 cells. *p < 0.05, **p < 0.01, ***p < 0.001.
4. Data with different superscripts in the same column were significantly different by using Student's t-test (p < 0.05).

TABLE 4
Effect of lovastatin on the growth of Hep G2 and Hep 3B cells.

| Incubation | Cell viability (% of control) | | | | | | IC₅₀ | |
|---|---|---|---|---|---|---|---|---|
| time | LOV (5 μM) | | | LOV (10 μM) | | | (μM) | |
| (hr) | Hep G2 | Hep 3B | Ratio | Hep G2 | Hep 3B | Ratio | Hep G2 | Hep 3B |
| 0 | 100.0 ± 1.0^{a} | 100.0 ± 1.3^{a} | 1.0 | 100.0 ± 2.3^{a} | 100.0 ± 1.8^{a} | 1.0 | — | — |
| 48 | 87.2 ± 3.1^{b} | 78.6 ± 4.7^{b} | 1.7* | 85.8 ± 2.7^{b} | 66.4 ± 0.8^{b} | 2.4*** | 97.2 | 63.6 |
| 72 | 80.9 ± 4.4^{c} | 58.8 ± 1.8^{c} | 2.2*** | 78.7 ± 5.3^{b} | 41.4 ± 1.5^{c} | 2.8*** | 62.3 | 6.8 |
| 96 | 63.2 ± 2.0^{d} | 56.4 ± 5.1^{c} | 1.2 | 55.1 ± 5.0^{c} | 31.7 ± 2.7^{d} | 1.5** | 13.1 | 5.9 |
| 120 | 55.1 ± 5.0^{d} | 20.3 ± 3.6^{d} | 1.8*** | 33.8 ± 3.8^{d} | 6.9 ± 1.3^{e} | 1.4*** | 6.2 | 1.3 |

1. Cells (4 × 10³/dish) were seeded in complete DMEM on 96 well culture plates for 24 hr. They were subsequently incubated in serum-free DMEM and treated with 5 μM and 10 μM lovastatin. Cell viability was determined by using the MTT assay. Each data represents mean ± SD (n = 4) from the same experiment.
2. IC₅₀, concentration of lovastatin required to inhibit cell viability by 50%.
3. Ratio, inhibition (%) on the growth of Hep 3B cells/inhibition (%) on the growth of Hep G2 cells. *p < 0.05, **p < 0.01, ***p < 0.001.
4. Data with different superscripts in the same column were significantly different by using Student's t-test (p < 0.05).

TABLE 5
Effect of lovastatin to inhibit cholesterol biosynthesis in Hep G2 and
Hep 3B cells as determined by incorporation of [2, ³H]acetate into
[³H]cholesterol.

| | | [³H]cholesterol | |
|---|---|---|---|
| Lovastatin | Incubation time | (10² dpm/10⁵ cells/hr) | |
| (μM) | (hr) | Hep G2 | Hep 3B |
| — | 24 | 61.09 | 59.64 |
| 1 | 24 | 30.14 (−50.7%) | 29.84 (−50.0%) |
| 1 | 48 | 70.43 (+15.3%) | 18.95 (−68.2%) |
| 5 | 24 | 16.39 (−73.2%) | 8.69 (−85.4%) |
| 5 | 48 | 31.46 (−48.5%) | 8.25 (−86.2%) |
| 10 | 2 | 5.00 (−91.8%) | 3.54 (−94.1%) |
| 10 | 6 | 3.67 (−94.0%) | 4.52 (−92.4%) |
| 10 | 12 | 4.07 (−93.3%) | 6.25 (−89.5%) |
| 10 | 24 | 7.83 (−87.2%) | 5.13 (−91.4%) |
| 10 | 48 | 18.71 (−69.4%) | 3.59 (−94.0%) |
| 50 | 24 | 2.54 (−95.8%) | 2.92 (−95.1%) |
| 50 | 48 | 9.13 (−85.1%) | 2.71 (−95.5%) |

1. Cells (1.5 × 10⁵/well) were seeded in complete DMEM on 12 well culture plates for 24 hr. They were subsequently incubated in serum-free DMEM and treated with various concentrations lovastatin.
[2, ³H]acetate (5 μCi/ml/well) was added 2 hr before the end of incubation times. Radioactivity was determined by using a liquid scintillation counter.
2. The percentages of inhibition were shown in parentheses.

TABLE 6
Effect of lovastatin alone and in combination with mevalonate on the growth of Hep G2 and Hep 3B cells.

| | | Cell viability (% of control) | | | | | |
|---|---|---|---|---|---|---|---|
| LOV | MVA | 24 hr | | 48 hr | | 72 hr | |
| (μM) | (mM) | Hep G2 | Hep 3B | Hep G2 | Hep 3B | Hep G2 | Hep 3B |
| 0 | 0 | 100.0 ± 4.1^{a} | 100.0 ± 7.0^{a} | 100.0 ± 3.4^{a} | 100.0 ± 5.5^{a} | 100.0 ± 2.1^{a} | 100.0 ± 5.5^{a} |
| 10 | 0 | — | 52.6 ± 2.6^{b} | — | 7.3 ± 0.5^{b} | — | 3.2 ± 1.8^{b} |
| 10 | 0.5 | — | 53.0 ± 2.3^{b} | — | 10.8 ± 2.4^{bc} | — | 8.4 ± 1.8^{c} |
| 10 | 1 | — | 56.9 ± 2.4^{c} | — | 15.4 ± 2.0^{c} | — | 9.1 ± 2.1^{c} |
| 10 | 2 | — | 58.8 ± 2.7^{c} | — | 21.1 ± 2.4^{d} | — | 14.2 ± 3.4^{c} |
| 50 | 0 | 57.8 ± 6.2^{b} | — | 12.7 ± 2.6^{b} | — | 4.0 ± 0.6^{b} | — |
| 50 | 0.5 | 64.0 ± 4.7^{b} | — | 26.8 ± 0.8^{c} | — | 21.3 ± 1.5^{c} | — |
| 50 | 1 | 71.1 ± 5.5^{c} | — | 29.9 ± 4.0^{c} | — | 32.3 ± 4.9^{d} | — |
| 50 | 2 | 75.7 ± 4.3^{c} | — | 37.6 ± 4.4^{d} | — | 37.7 ± 3.8^{d} | — |
| 1. Cells (5 × 10³/well) were seeded in complete DMEM on 96 well culture plats for 24 hr. They were subsequently incubated in serum-free DMEM and treated with 50 μM lovastatin (LOV) (Hep G2) and 10 μM lovastatin (Hep 3B). Following 48 hr incubation, cells were changed to fresh DMEM containing lovastatin and various concentrations of mevalonate (MVA) for an other 24, 48 and 72 hr. | | | | | | | |
| # Cell viability was determined by using the MTT assay. Each data represents mean ± SD (n = 6) from the same experiment. | | | | | | | |

2. Data with different superscripts in the same column were significantly different by using Student's t-test (p < 0.05).

TABLE 7
Effect of lovastatin alone and in combination with mevalonate on the growth
of Hep G2 and Hep 3B cells.

| | | Cell viability (1 × 10³ cells) | | | | | |
|---|---|---|---|---|---|---|---|
| LOV | MVA | 24 hr | | 48 hr | | 72 hr | |
| (μM) | (mM) | Hep G2 | Hep 3B | Hep G2 | Hep 3B | Hep G2 | Hep 3B |
| 0 | 0 | 18.3 ± 0.7^{a} | 10.0 ± 0.7^{a} | 28.7 ± 2.4^{a} | 13.3 ± 0.7^{a} | 43.5 ± 1.7^{a} | 17.8 ± 1.5^{a} |
| 10 | 0 | — | 5.3 ± 0.1^{b} | — | 1.0 ± 0.1^{b} | — | 0.6 ± 0.5^{b} |
| 10 | 0.5 | — | 5.3 ± 0.2^{b} | — | 1.4 ± 0.3^{b} | — | 1.5 ± 0.6^{bc} |
| 10 | 1 | — | 5.7 ± 0.1^{c} | — | 2.1 ± 0.3^{c} | — | 1.6 ± 0.5^{cd} |
| 10 | 2 | — | 5.9 ± 0.2^{c} | — | 2.8 ± 0.3^{d} | — | 2.5 ± 0.9^{c} |
| 50 | 0 | 10.6 ± 0.7^{b} | — | 3.6 ± 1.6^{b} | — | 1.7 ± 0.5^{b} | — |
| 50 | 0.5 | 11.7 ± 0.5^{b} | — | 7.7 ± 0.5^{c} | — | 9.3 ± 1.3^{c} | — |
| 50 | 1 | 13.1 ± 0.8^{c} | — | 8.6 ± 2.6^{cd} | — | 14.1 ± 4.1^{d} | — |
| 50 | 2 | 13.9 ± 0.6^{c} | — | 10.8 ± 2.7^{d} | — | 16.4 ± 3.2^{d} | — |
| 1. Cells (5 × 10³/well) were seeded in complete DMEM on 96 well culture plats for 24 hr. They were subsequently incubated in serum-free DMEM and treated with 50 μM lovastatin (LOV) (Hep G2) and 10 μM lovastatin (Hep 3B). Following 48 hr incubation, cells were changed to fresh DMEM containing lovastatin and various concentrations of mevalonate for another 24, 48 and 72 hr. | | | | | | | |
| # Cell number was determined by using the MTT assay. Each data represents mean ± SD (n = 6) from the same experiment. | | | | | | | |

2. Data with different superscripts in the same column were significantly different by using Student's t-test (p < 0.05).

TABLE 8
Effect of taxol on the growth of Hep G2 and Hep 3B cells.

| | Cell viability (% of control) | | | | | |
|---|---|---|---|---|---|---|
| Taxol | 48 hr | | | 72 hr | | |
| (nM) | Hep G2 | Hep 3B | Ratio | Hep G2 | Hep 3B | Ratio |
| 0 | 100.0^{a} | 100.0^{a} | 1.0 | 100.0^{a} | 100.0^{a} | 1.0 |
| 2 | 94.5 ± 5.4^{b} | 93.1 ± 1.3^{b} | 1.3* | 89.2 ± 2.6^{b} | 94.0 ± 3.6^{b} | 0.6* |
| 5 | 72.9 ± 4.0^{c} | 91.6 ± 0.5^{b} | 0.3** | 63.7 ± 2.3^{c} | 91.7 ± 3.7^{bc} | 0.2** |
| 10 | 64.2 ± 3.8^{c} | 87.1 ± 1.5^{c} | 0.4*** | 39.0 ± 0.5^{d} | 88.5 ± 2.4^{bc} | 0.2*** |
| 20 | 53.5 ± 1.1^{d} | 85.4 ± 0.9^{cd} | 0.3*** | 35.4 ± 3.6^{d} | 85.6 ± 1.1^{c} | 0.2*** |
| 50 | 47.0 ± 2.3^{e} | 83.5 ± 0.9^{d} | 0.3*** | 33.9 ± 3.7^{de} | 71.2 ± 4.2^{d} | 0.4*** |
| 100 | 34.6 ± 9.1^{ef} | 74.9 ± 1.8^{ef} | 0.4** | 19.5 ± 4.6^{e} | 52.7 ± 2.8^{ef} | 0.6** |
| 200 | 35.4 ± 5.5^{f} | 64.7 ± 4.6^{fg} | 0.5** | 15.6 ± 5.8^{efg} | 47.0 ± 1.0^{fg} | 0.6** |
| 500 | 29.1 ± 4.2^{f} | 56.2 ± 0.8^{gh} | 0.6** | 11.0 ± 5.0^{fg} | 32.2 ± 4.7^{g} | 0.8** |
| 1000 | 15.0 ± 3.9^{f} | 55.3 ± 0.5^{h} | 0.5** | 9.9 ± 4.9^{g} | 24.6 ± 2.9^{h} | 0.8** |
| IC₅₀ | 40.8 ± 7.0 | 1118.6 ± 10.1*** | | 7.8 ± 1.0 | 147.4 ± 14.2*** | |

1. Cells (8 × 10³/well) were seeded in complete DMEM on 96 well culture plates for 24 hr. They were subsequently incubated in serum-free DMEM and treated with various concentrations of taxol for 48 hr and 72 hr. Cell viability was determined by using the MTT assay. Each data represents mean ± SD (n = 6) from three experiments.
2. IC₅₀, concentration of taxol required to inhibit cell viability by 50%.
3. Ratio, inhibition (%) on the growth of Hep 3B cells inhibition (%) on the growth of Hep G2 cells. *p < 0.05, **p < 0.01, ***p < 0.001
4. Data with different superscripts in the same column were significantly different by using Student's t-test (p <0.05).

TABLE 9
Effect of taxol on the growth of Hep G2 and Hep 3B cells

| Incubation | Cell viability (% of control) | | | | | | IC₅₀ | |
|---|---|---|---|---|---|---|---|---|
| time | Taxol (2 nM) | | Taxol (5 nM) | | Taxol (10 nM) | | (nM) | |
| (hr) | Hep G2 | Hep 3B | Hep G2 | Hep 3B | Hep G2 | Hep 3B | Hep G2 | Hep 3B |
| 0 | 100.0 ± 6.1^{a} | 100.0 ± 1.8^{a} | 100.0 ± 1.0^{a} | 100.0 ± 1.3^{a} | 100.0 ± 2.3^{a} | 100.0 ± 1.8^{a} | — | — |
| 48 | 88.0 ± 1.9^{b} | 97.0 ± 1.5^{ab} | 74.7 ± 2.3^{b} | 97.5 ± 3.1^{ab} | 58.1 ± 8.1^{b} | 89.1 ± 3.0^{b} | 12.4 | 45.9 |
| 72 | 89.2 ± 2.6^{b} | 94.0 ± 3.6^{ab} | 63.7 ± 2.3^{bc} | 91.7 ± 3.7^{b} | 39.0 ± 0.5^{c} | 88.5 ± 2.4^{b} | 7.8 | 43.5 |
| 96 | 66.6 ± 3.5^{c} | 94.1 ± 7.9^{ab} | 36.2 ± 5.5^{d} | 92.4 ± 4.8^{abc} | 22.7 ± 4.1^{d} | 82.3 ± 6.6^{c} | 3.6 | 28.2 |
| 120 | 65.1 ± 0.9^{c} | 94.2 ± 2.5^{b} | 33.8 ± 0.6^{d} | 82.7 ± 3.7^{c} | 19.0 ± 3.4^{d} | 71.9 ± 4.6^{d} | 3.5 | 17.8 |

1. Cells (4 × 10³/well) were seeded in complete DMEM on 96 well culture plates for 24 hr. They were subsequently incubated in serum-free DMEM and treated with various concentrations of taxol. Cell viability was determined by using the MTT assay. Each data represents mean ± SD (n = 4) from the same experiment.
2. IC₅₀, concentration of taxol required to inhibit cell viability by 50%.
3. Ratio, inhibition (%) on the growth of Hep 3B cells/inhibition (%) on the growth of Hep G2 cells.
4. Data with different superscripts in the same column were significantly different by using Student's t-test (p < 0.05).

TABLE 10
Effects of combined treatment of lovastatin and taxol on the growth
of Hep G2 and Hep 3B cells.

| | | Cell viability | | |
|---|---|---|---|---|
| Lovastatin | Taxol | (% of control) | | |
| (μM) | (nM) | Hep G2 | Hep 3B | Ratio |
| 0 | 0 | 100.0 ± 8.9^{a} | 100.0 ± 5.4^{a} | 1.0 |
| 0 | 2 | 72.4 ± 6.9^{b} | 92.6 ± 1.9^{a} | 0.3** |
| 0 | 5 | 49.3 ± 9.9^{c} | 79.5 ± 4.6^{b} | 0.4** |
| 0 | 10 | 24.8 ± 2.5^{d} | 60.3 ± 3.4^{c} | 0.5*** |
| 10 | 0 | 51.9 ± 4.4^{c} | 29.8 ± 1.2^{d} | 1.5** |
| 10 | 2 | 52.1 ± 3.7^{c} | 32.1 ± 2.4^{d} | 1.4** |
| 10 | 5 | 26.8 ± 1.9^{d} | 29.9 ± 1.6^{d} | 1.0 |
| 10 | 10 | 10.1 ± 2.4^{e} | 15.1 ± 0.8^{e} | 0.9* |

1. Cells (5 × 10³/well) were seeded in complete DMEM on 96 well culture plates for 24 hr. They were subsequently incubated in serum-free DMEM and simultaneously treated with lovastatin and taxol for 96 hr.
Cell viability was determined by using the MTT assay. Each data represents mean ± SD (n = 6) from the same experiment.
2. Ratio, inhibition (%) on the growth of Hep 3B/inhibition (%) on the growth of Hep G2.
*p<0.05,
**p<0.01,
***p<0.001.
3. Data with different superscripts in the column were significantly different by using Student's t-test (p<0.05).

TABLE 11
Effects of taxol on the growth of Hep G2 and Hep 3B cells after 48 hr
lovastatin treatment.

| | | Cell viability | | |
|---|---|---|---|---|
| Lovastatin | Taxol | (% of control) | | |
| (μM) | (nM) | Hep G2 | Hep 3B | Ratio |
| 0 | 0 | 100.0 ± 4.5^{a} | 100.0 ± 2.3^{a} | 1.0 |
| 0 | 2 | 92.2 ± 1.5^{b} | 102.4 ± 4.7^{a} | — |
| 0 | 5 | 61.5 ± 2.2^{c} | 87.1 ± 4.1^{b} | 0.3*** |
| 0 | 10 | 57.9 ± 3.3^{ce} | 76.3 ± 1.3^{c} | 0.6*** |
| 5 | 0 | 85.8 ± 4.5 | 49.8 ± 7.8 | 3.5 |
| 5 | 2 | 77.9 ± 1.9 | 44.0 ± 2.8 | 2.5 |
| 5 | 5 | 56.6 ± 2.7 | 54.2 ± 5.6 | 1.1 |
| 5 | 10 | 42.7 ± 2.2 | 25.8 ± 1.1 | 1.3 |
| 10 | 0 | 69.1 ± 3.1^{d} | 45.7 ± 3.1^{d} | 1.9*** |
| 10 | 2 | 60.4 ± 4.6^{ce} | 25.9 ± 3.8^{c} | 1.9*** |
| 10 | 5 | 50.8 ± 3.1^{e} | 31.2 ± 1.4^{e} | 1.4*** |
| 10 | 10 | 41.9 ± 5.7^{f} | 12.3 ± 4.1^{f} | 1.5** |
| 50 | 0 | 14.6 ± 1.3 | 2.9 ± 4.1 | 1.1 |
| 50 | 2 | 11.9 ± 1.4 | 0.0 ± 2.9 | 1.1 |
| 50 | 5 | 10.7 ± 0.7 | 0.0 ± 1.8 | 1.1 |
| 50 | 10 | 6.9 ± 2.5 | 0.0 ± 1.0 | 1.1 |

1.Cells (5 × 10³/well) were seeded in complete DMEM on 96 well culture plates for 24 hr. They were subsequently incubated in serum-free DMEM and treated with various concentrations of lovastatin.
Following 48 hr incubation, cells were changed to fresh DMEM containing various concentrations of taxol for another 48 hr. Cell viability was determined by using the MTT assay. Each data represents mean ± SD (n = 6) from the same experiment.
2. Ratio, inhibition (%) on the growth of Hep 3B cells / inhibition (%) on the growth of Hep G2 cells.
*p>0.05,
**p<0.01,
***p<0.001.
3. Data with different superscripts in the column were significantly different by using Student's t-test (p<0.05).

TABLE 12
Effects of taxol on the growth of Hep G2 and Hep 3B cells after 72 hr
lovastatin treatment.

| | | Cell viability | | |
|---|---|---|---|---|
| Lovastatin | Taxol | (% of control) | | |
| (μM) | (nM) | Hep G2 | Hep 3B | Ratio |
| 0 | 0 | 100.0 ± 0.7^{a} | 100.0 ± 2.8^{a} | 1.0 |
| 0 | 2 | 85.1 ± 1.9^{b} | 94.1 ± 5.6^{ab} | 0.4*** |
| 0 | 5 | 64.2 ± 1.2^{c} | 86.9 ± 1.3^{b} | 0.4*** |
| 0 | 10 | 60.5 ± 3.2^{c} | 85.6 ± 5.6^{b} | 0.4** |
| 5 | 0 | 81.3 ± 3.8 | 27.7 ± 2.0 | 3.9 |
| 5 | 2 | 60.4 ± 3.5 | 19.4 ± 3.6 | 2.0 |
| 5 | 5 | 54.1 ± 3.9 | 18.7 ± 5.8 | 1.8 |
| 5 | 10 | 41.0 ± 1.9 | 9.3 ± 7.6 | 1.5 |
| 10 | 0 | 54.7 ± 5.9^{cd} | 20.4 ± 7.0^{c} | 1.8** |
| 10 | 2 | 52.9 ± 3.6^{cd} | 17.4 ± 4.2^{cd} | 1.8*** |
| 10 | 5 | 43.7 ± 2.7^{e} | 18.1 ± 0.8^{c} | 1.5*** |
| 10 | 10 | 35.8 ± 5.0^{e} | 8.9 ± 2.8^{d} | 1.4** |

1. Cells (5 × 10³/well) were seeded in complete DMEM on 96 well culture plates for 24 hr. They were subsequently incubated in serum-free DMEM and treated with various concentrations of lovastatin. Following 72 hr incubation, cells were changed to fresh DMEM containing various concentrations of taxol for another 48 hr.
Cell viability was determined by using the MTT assay. Each data represents mean ± SD (n = 6) from the same experiment.
2. Ratio, inhibition (%) on the growth of Hep 3B cells inhibition (%) on the growth of Hep G2 cells.
*p<0.05,
**p<0.01
***p<0.001
3. Data with different superscripts in the column were signifcantly different by using Student's t-test (p<0.05).

TABLE 13
Effects of taxol on the growth of Hep G2 and Hep 3B cells after 72 hr
lovastatin treatment.

| | | Cell viability | | |
|---|---|---|---|---|
| Lovastatin | Taxol | (% of control) | | |
| (μM) | (nM) | Hep G2 | Hep 3B | Ratio |
| 0 | 0 | 100.0 ± 8.9^{a} | 100.0 ± 5.2^{a} | 1.0 |
| 0 | 2 | 77.7 ± 2.6^{b} | 91.1 ± 1.1^{b} | 0.4* |
| 0 | 5 | 65.8 ± 1.3^{c} | 96.5 ± 0.6^{a} | 0.1*** |
| 0 | 10 | 63.6 ± 8.4^{cd} | 78.0 ± 0.4^{c} | 0.6** |
| 5 | 0 | 67.2 ± 0.5 | 13.0 ± 3.0 | 2.7 |
| 5 | 2 | 62.5 ± 1.4 | 20.5 ± 3.3 | 2.1 |
| 5 | 5 | 62.6 ± 0.9 | 46.5 ± 6.9 | 2.2 |
| 5 | 10 | 53.8 ± 4.1 | 14.2 ± 2.7 | 1.9 |
| 10 | 0 | 48.0 ± 2.9^{de} | 14.7 ± 3.6^{d} | 1.6*** |
| 10 | 2 | 47.5 ± 5.5^{de} | 11.1 ± 2.0^{d} | 1.7*** |
| 10 | 5 | 47.2 ± 3.0^{de} | 10.0 ± 4.3^{d} | 1.7*** |
| 10 | 10 | 40.6 ± 3.8^{e} | 9.0 ± 0.9^{d} | 1.5*** |

1. Cells (5 × 10³/well) were seeded in complete DMEM on 96 well culture plates for 24 hr. They were subsequently incubated in serum-free DMEM and treated with various concentrations of lovastatin.
Following 72 hr incubation, cells were changed to fresh DMEM containing various concentrations of taxol for another 72 hr. Cell viability was determined by using the MTT assay. Each data represents mean ± SD (n = 6) from the same experiment.
2. Ratio, inhibition (%) on the growth of Hep 3B cells / inhibition (%) on the growth of Hep G2 cells.
*p<0.05,
**p<0.01,
***p<0.001.
3. Data with different superscripts in the column were significantly different by using Student's t-test (p<0.05).

TABLE 14
Effect of doxorubicin on the growth of Hep G2 and Hep 3B cells.

| | Cell viability (% of control) | | | | | |
|---|---|---|---|---|---|---|
| DOX | 48 hr | | | 72 hr | | |
| (μM) | Hep G2 | Hep 3B | Ratio | Hep G2 | Hep 3B | Ratio |
| 0 | 100.0 ± 1.0^{a} | 100.0 ± 2.3^{a} | 1.0 | 100.0 ± 1.6^{a} | 100.0 ± 0.8^{a} | 1.0 |
| 0.1 | 100.1 ± 5.3^{a} | 99.9 ± 7.8^{a} | — | 99.9 ± 7.5^{a} | 100.3 ± 5.8^{a} | — |
| 0.25 | 70.7 ± 0.7^{b} | 79.8 ± 4.2^{b} | 0.7* | 66.9 ± 5.3^{b} | 75.6 ± 1.8^{b} | 0.7 |
| 0.5 | 42.8 ± 3.7^{c} | 66.7 ± 1.6^{c} | 0.6*** | 46.7 ± 4.0^{c} | 62.1 ± 6.9^{c} | 0.7* |
| 1 | 29.1 ± 1.7^{d} | 59.3 ± 4.1^{d} | 0.6*** | 20.0 ± 1.1^{d} | 48.9 ± 8.4^{c} | 0.6** |
| 2 | 16.7 ± 2.4^{e} | 40.1 ± 3.0^{e} | 0.7*** | 1.9 ± 0.8^{e} | 9.9 ± 2.7^{d} | 0.9** |
| IC₅₀ | 0.4 | 1.5 | | 0.5 | 1.0 | |

1. Cells (8 × 10³/well) were seeded in complete DMEM on 96 well culture plates for 24 hr. They were subsequently incubated in serum-free DMEM and treated with various concentrations of doxorubicin (DOX) for 48 hr and 72 hr. Cell viability was determined by using the MTT assay. Each data represents mean ± SD (n = 6) from the same experiment.
2. IC₅₀, concentration of doxorubicin required to inhibit cell viability by 50%.
3. Ratio, inhibition (%) on the growth of Hep 3B cells inhibition (%) on the growth of Hep G2 cells. *p < 0.05, **p < 0.01, ***p < 0.001
4. Data with different superscripts in the same column were significantly different by using Student's t-test (p < 0.05).

TABLE 15
Effects of several Chinese herbs on the growth Hep G2
and Hep 3B cells.

| Treatment | | | | | | |
|---|---|---|---|---|---|---|
| Conc. | 48 hr | | | 72 hr | | |
| (μg/ml) | Hep G2 | Hep 3B | Ratio | Hep G2 | Hep 3B | Ratio |
| DO-M1 | | | | | | |
| 10 | 131.4 ± 21.1 | 109.2 ± 0.5 | — | 103.3 ± 10.6 | 87.2 ± 12.7 | — |
| 20 | 86.9 ± 7.3 | 74.3 ± 5.4 | 2.0 | 57.9 ± 11.0 | 35.6 ± 8.2 | 1.5 |
| 40 | 44.1 ± 27.7 | 43.9 ± 7.5 | 1.0 | 35.0 ± 5.1 | 10.7 ± 15.1 | 1.4 |
| SM-M1 | | | | | | |
| 10 | 108.9 ± 14.1 | 102.6 ± 5.4 | — | 100.5 ± 1.6 | 108.3 ± 25.1 | — |
| 20 | 78.5 ± 3.6 | 96.7 ± 13.4 | 0.2 | 72.4 ± 2.3 | 96.2 ± 27.2 | 0.1 |
| 40 | 59.7 ± 6.5 | 86.4 ± 9.3 | 0.3 | 52.3 ± 1.8 | 81.4 ± 12.6 | 0.4 |
| PL-M1 | | | | | | |
| 10 | 89.3 ± 0.2 | 87.2 ± 0.2 | 1.2 | 63.1 ± 6.4 | 53.2 ± 5.7 | 1.3 |
| 20 | 77.3 ± 0.1 | 66.5 ± 8.8 | 1.5 | 42.3 ± 18.8 | 33.5 ± 11.2 | 1.2 |
| 40 | 70.5 ± 0.1 | 67.5 ± 3.0 | 1.1 | 39.2 ± 12.9 | 29.9 ± 5.4 | 1.2 |
| LC-M1 | | | | | | |
| 10 | 114.7 ± 21.1 | 125.0 ± 8.7 | 0.6 | 118.8 ± 12.1 | 127.9 ± 17.5 | 0.7 |
| 20 | 127.5 ± 7.3 | 127.8 ± 7.1 | 1.0 | 120.8 ± 11.8 | 131.0 ± 12.7 | 0.7 |
| 40 | 119.9 ± 27.7 | 134.2 ± 12.5 | 0.6 | 130.1 ± 9.9 | 136.8 ± 7.8 | 0.8 |
| CT-M1 | | | | | | |
| 10 | 127.2 ± 14.1 | 126.6 ± 4.7 | 1.0 | 117.5 ± 11.5 | 131.2 ± 4.4 | 0.5 |
| 20 | 135.5 ± 3.6 | 129.0 ± 11.4 | 1.2 | 126.5 ± 13.6 | 134.1 ± 6.5 | 0.8 |
| 40 | 130.9 ± 6.5 | 136.1 ± 6.7 | 0.9 | 143.4 ± 8.4 | 149.1 ± 6.0 | 0.8 |
| 1. Cells (8 × 10³/well) were seeded in complete DMEM on 96 well culture plates for 24 hr. They were subsequently incubated in serum-free DMEM and treated with various concentrations of methanolic extracts of Dalberdia odorifera (DO-M1), Saliva miltiorrhiza (SM-M1), Paeonia lactylora (PL-M1) Ligusticum chuanxiong (LC-M1) and Carthamus tinctorius | | | | | | |
| # L. (CL-M1) for 48 hr and 72 hr, respectively. Cell viability was determined by using the MTT assay. Each data represents mean ± SD (n = 6) from two experiments. | | | | | | |

2. Ratio, the ratio of DO-M1, SM-M1 and PL-M1 treatment were inhibition (%) on the growth of Hep 3B cells/inhibition (%) on the growth of Hep G2 cells. The ratio of LC-M1 and CT-M1 treatment were proliferation (%) on the growth of Hep G2 cells/proliferation (%) on the growth of Hep 3B cells.

TABLE 16
Effect of Cordyceps sinensis on the growth of
Hep G2 and Hep 3B cells.

| | Cell viability | | | |
|---|---|---|---|---|
| | (% of control) | | | |
| CS | 48 hr | | 72 hr | |
| (μg/ml) | Hep G2 | Hep 3B | Hep G2 | Hep 3B |
| 0 | 100.0 ± 1.1^{a} | 100.0 ± 6.9^{a} | 100.0 ± 7.7^{a} | 100.0 ± 7.6^{a} |
| 10 | 91.1 ± 2.4^{b} | 97.8 ± 6.4^{a} | 115.1 ± 6.6^{b} | 95.8 ± 2.5^{b} |
| 20 | 88.0 ± 3.4^{b} | 100.9 ± 4.6^{a} | 120.3 ± 3.8^{bc} | 105.0 ± 3.3^{c} |
| 40 | 95.1 ± 3.9^{a} | 110.0 ± 2.8^{b} | 128.2 ± 3.7^{c} | 107.1 ± 4.2^{c} |
| 100 | 90.8 ± 7.8^{ab} | 96.0 ± 3.9^{a} | 127.7 ± 3.2^{bc} | 99.4 ± 0.8^{a} |

1. The water- and ethanol-soluble fraction (CS-KY2-NH1-El) of Cordyceps sinensis (CS) was used.
2. Cells (8 × 10³/well) were seeded in complete DMEM on 96 well culture plates for 24 hr. They were subsequently incubated in serum-free DMEM and treated with various concentrations of CS for 48 hr and 72 hr.
Cell viability was determined by using the MTT assay. Each data represents mean ± SD (n = 6) from the same experiment.
3. Data with different superscripts in the same column were significantly different by using Student's t-test (p<0.05).

TABLE 17
Effect of Ganoderma spp. on the growth of Hep G2 and Hep 3B
cells.

| | Cell viability (% of control) | | | | | |
|---|---|---|---|---|---|---|
| GL-M1 | 48 hr | | | 72 hr | | |
| (μg/ml) | Hep G2 | Hep 3B | Ratio | Hep G2 | Hep 3B | Ratio |
| 0 | 100.0 ± 2.5^{a} | 100.0 ± 1.8^{a} | 1.0 | 100.0 ± 1.4^{a} | 100.0 ± 2.1^{a} | 1.0 |
| 2.5 | 131.2 ± 4.9^{b} | 104.8 ± 3.4^{a} | — | 108.0 ± 3.8^{b} | 92.1 ± 2.4^{b} | — |
| 5 | 137.5 ± 3.7^{b} | 99.1 ± 1.9^{a} | — | 105.4 ± 5.7^{a} | 93.7 ± 4.3^{a} | — |
| 10 | 119.3 ± 4.8^{c} | 107.8 ± 2.2^{b} | — | 70.1 ± 2.7^{c} | 68.0 ± 4.3^{c} | 1.1 |
| 15 | 92.8 ± 3.4^{d} | 57.7 ± 2.8^{c} | 5.9*** | 67.5 ± 1.2^{c} | 22.1 ± 2.4^{d} | 2.4*** |
| 20 | 92.9 ± 3.7^{d} | 45.3 ± 3.5^{d} | 7.7*** | 54.2 ± 1.0^{d} | 3.1 ± 2.5^{e} | 2.1*** |
| 40 | 79.1 ± 4.8^{e} | 7.1 ± 4.5^{e} | 4.4*** | 27.0 ± 4.1^{e} | 0.0 ± 3.2^{e} | 1.4*** |
| IC₅₀ | 81.2 | 18.1 | | 23.1 | 12.0 | |

1. The methanolic extracts of Ganoderma spp. (GL-M1) were used.
2. Cells (8 × 10³/well) were seeded in complete DMEM on 96 well culture plates for 24 hr. They were subsequently incubated in serum-free DMEM and treated with various concentrations of GL-M1 for 48 hr and 72 hr. Cell viability was determined by using the MTT assay. Each data represents mean ± SD (n = 6) from two experiments.
3. IC₅₀, concentration of GL-M1 required to inhibit cell viability by 50%.
4. Ratio, inhibition (%) on the growth of Hep 3B cells/inhibition (%) on the growth of Hep G2 cells. *p < 0.05, **p < 0.01, ***p < 0.001.
5. Data with different superscripts in the same column were significantly different by using Student's t-test (p < 0.05).

TABLE 18
Effect of caffeic acid phenethyl ester on the growth of Hep G2 and
Hep 3B cells.

| | Cell viability (% of control) | | | | | |
|---|---|---|---|---|---|---|
| CAPE | 48 hr | | | 72 hr | | |
| (μM) | Hep G2 | Hep 3B | Ratio | Hep G2 | Hep 3B | Ratio |
| 0 | 100.0 ± 1.0^{a} | 100.0 ± 2.3^{a} | 1.0 | 100.0 ± 2.2^{a} | 100.0 ± 2.7^{a} | 1.0 |
| 2.5 | 95.2 ± 3.5^{a} | 96.7 ± 1.4^{a} | 0.7 | 81.0 ± 3.7^{b} | 79.2 ± 5.2^{b} | 1.1 |
| 5 | 71.1 ± 1.8^{b} | 87.1 ± 1.8^{b} | 0.4*** | 42.3 ± 2.5^{c} | 70.8 ± 2.6^{b} | 0.5*** |
| 10 | 47.4 ± 3.0^{c} | 73.2 ± 1.2^{c} | 0.5*** | 21.2 ± 5.0^{d} | 47.6 ± 1.9^{c} | 0.7** |
| 25 | 42.6 ± 1.3^{c} | 38.2 ± 4.7^{d} | 1.1 | 12.5 ± 3.0^{d} | 12.5 ± 4.8^{d} | 1.0 |
| 50 | 7.5 ± 0.6^{d} | 20.5 ± 4.6^{e} | 0.9** | 1.9 ± 1.8^{e} | 0.0 ± 3.5^{e} | 1.0 |
| IC₅₀ | 9.5 | 19.9 | | 4.5 | 9.5 | |

1. Cells (8 × 10³/well) were seeded in complete DMEM on 96 well culture plates for 24 hr. They were subsequently incubated in serum-free DMEM and treated with various concentrations of caffeic acid phenethyl ester (CAPE) for 48 hr and 72 hr. Cell viability was determined by using the MTT assay. Each data represents mean ± SD (n = 6) from the same experiment.
2. IC₅₀, concentration of CAPE required to inhibit cell viability by 50%.
3. Ratio, inhibition (%) on the growth of Hep 3B cells/inhibition (%) on the growth of Hep G2 cells. *p < 0.05, **p < 0.01, ***p < 0.001.
4. Data with different superscripts in the same column were significantly different by using Student's t-test (p < 0.05).

TABLE 19
Effect of caffeic acid 1-octyl ester on the growth of Hep G2 and Hep 3B cells.

| | Cell viability (% of control) | | | | | |
|---|---|---|---|---|---|---|
| CAO | 48 hr | | | 72 hr | | |
| (μM) | Hep G2 | Hep 3B | Ratio | Hep G2 | Hep 3B | Ratio |
| 0 | 100.0 ± 1.6^{a} | 100.0 ± 7.3^{d} | 1.0 | 100.0 ± 5.4^{a} | 100.0 ± 4.8^{a} | 1.0 |
| 1.25 | 103.9 ± 4.6^{a} | 97.8 ± 4.1^{a} | — | 102.7 ± 0.4^{b} | 89.8 ± 0.6^{b} | — |
| 2.5 | 58.1 ± 5.9^{b} | 69.6 ± 6.1^{b} | 0.7 | 53.3 ± 6.3^{c} | 56.3 ± 5.6^{c} | 0.9 |
| 5 | 44.2 ± 6.6^{b} | 52.0 ± 1.8^{c} | 0.9** | 18.1 ± 2.1^{d} | 16.3 ± 2.6^{d} | 1.0 |
| 10 | 5.8 ± 3.9^{c} | 21.7 ± 3.9^{d} | 0.8 | 0.0 ± 7.1^{e} | 2.4 ± 1.6^{e} | 1.0 |
| 25 | 0.0 ± 1.3^{c} | 3.9 ± 1.3^{e} | 1.0* | 0.0 ± 1.4^{e} | 0.0 ± 3.4^{e} | 1.0 |
| IC₅₀ | 4.0 | 5.3 | | 2.7 | 2.9 | |

1. Cells (8 × 10³/well) were seeded in complete DMEM on 96 well culture plates for 24 hr. They were subsequently incubated in serum-free DMEM and treated with various concentrations of caffeic acid 1-octyl ester (CAO) for 48 hr and 72 hr. Cell viability was determined by using the MTT assay. Each data represents mean ± SD (n = 6) from the same experiment.
2. IC50, concentration of CAO required to inhibit cell viability by 50%.
3. Ratio, inhibition (%) on the growth of Hep 3B cells inhibition (%) on the growth of Hep G2 cells. *p < 0.05, **p < 0.01, ***p < 0.001
4. Data with different superscripts in the same column were significantly different by using Student's t-test (p < 0.05).

TABLE 20
Effect of farnesyltransferase inhibitor on the growth of Hep G2 and Hep 3B cells.

| | Cell viability (% of control) | | | | | |
|---|---|---|---|---|---|---|
| FTI | 48 hr | | | 72 hr | | |
| (nM) | Hep G2 | Hep 3B | Ratio | Hep G2 | Hep 3B | Ratio |
| 0 | 100.0^{a} | 100.0^{a} | 1.0 | 100.0^{a} | 100.0^{a} | 1.0 |
| 5 | 95.7 ± 0.8^{b} | 99.6 ± 0.3^{a} | 0.1** | 92.3 ± 3.4^{b} | 98.7 ± 1.4^{ab} | 0.2* |
| 10 | 91.9 ± 3.4^{b} | 99.4 ± 0.5^{a} | 0.1* | 91.1 ± 3.5^{b} | 98.2 ± 1.7^{ab} | 0.2* |
| 20 | 89.4 ± 4.5^{bc} | 96.5 ± 2.7^{ab} | 0.3 | 89.0 ± 3.3^{b} | 96.5 ± 1.5^{b} | 0.3* |
| 50 | 87.1 ± 1.6^{cd} | 95.4 ± 3.6^{ab} | 0.4* | 88.6 ± 3.6^{bc} | 95.8 ± 1.6^{b} | 0.4* |
| 100 | 84.0 ± 5.7^{de} | 92.6 ± 1.6^{b} | 0.5 | 86.3 ± 1.7^{bc} | 90.0 ± 6.1^{bc} | 0.7 |
| 200 | 78.8 ± 1.8^{e} | 88.7 ± 0.2^{c} | 0.5*** | 82.8 ± 1.5^{c} | 82.4 ± 4.8^{c} | 1.0 |
| 500 | 70.5 ± 0.3^{f} | 86.5 ± 2.4^{cd} | 0.5*** | 65.3 ± 3.9^{d} | 81.4 ± 4.7^{cd} | 0.5* |
| 1000 | 53.8 ± 0.6^{g} | 85.8 ± 2.8^{d} | 0.3*** | 50.4 ± 4.1^{e} | 74.5 ± 2.5^{de} | 0.5*** |
| 2000 | 51.8 ± 1.3^{g} | 82.4 ± 1.5^{d} | 0.4*** | 30.9 ± 6.2^{f} | 70.0 ± 3.7^{e} | 0.4*** |

1. Cells (8 × 10³/well) were seeded in complete DMEM on 96 well culture plates for 24 hr. They were subsequently incubated in serum-free DMEM and treated with various concentrations of farnesyltransferase inhibitor (FTI) for 48 hr and 72 hr. Cell viability was determined by using the MTT assay. Each data represents mean ± SD (n = 4) from two experiments.
2. Ratio, inhibition (%) on the growth of Hep 3B inhibition (%) on the growth of Hep G2. *p < 0.05, **p < 0.01, ***p < 0.001.
3. Data with different superscripts in the column were significantly different by using Student's t-test (p < 0.05).

TABLE 21
Effects of combined treatment of lovastatin and Ganoderma spp. on
the growth of Hep G2 and Hep 3B cells.

| | | Cell viability | | |
|---|---|---|---|---|
| Lovastatin | GL-Ml | (% of control) | | |
| (μM) | (μg/ml) | Hep G2 | Hep 3B | Ratio |
| 0 | 0 | 100.0 ± 9.0^{a} | 100.0 ± 1.9^{a} | 1.0 |
| 0 | 5 | 117.8 ± 6.5^{b} | 105.2 ± 8.5^{a} | — |
| 0 | 10 | 67.0 ± 4.1^{c} | 101.0 ± 3.4^{a} | — |
| 0 | 15 | 40.9 ± 4.3^{d} | 18.4 ± 4.7^{b} | 1.4** |
| 0 | 20 | 16.5 ± 0.4^{e} | 9.7 ± 3.1^{c} | 1.1* |
| 10 | 0 | 52.4 ± 5.5^{f} | 27.5 ± 8.2^{d} | 1.5** |
| 10 | 5 | 39.5 ± 4.5^{d} | 24.0 ± 1.8^{bd} | 1.3** |
| 10 | 10 | 2.9 ± 1.1^{g} | 21.0 ± 1.3^{bd} | 0.8*** |
| 10 | 15 | 0.0 ± 0.7ⁿ | 4.6 ± 0.5^{e} | 1.0*** |
| 10 | 20 | 0.0 ± 0.2^{h} | 4.0 ± 1.0^{e} | 1.0** |

1. Cells (5 × 10³/well) were seeded in complete DMEM on 96 well culture plates for 24 hr. They were subsequently incubated in serum-free DMEM and simultaneously treated with lovastatin and methanolic extracts of Ganoderma spp.
(GL-M1) for 96 hr. Cell viability was determined by using the MTT assay. Each data represents mean ± SD (n = 6) from the same experiment.
2. Ratio, inhibition (%) on the growth of Hep 3B inhibition (%) on the growth of Hep G2.
*p<0.05,
**p<0.01,
***p<0.001
3. Data with different superscripts in the column were significantly different by using Student's t-test (p<0.05).

TABLE 22
Effects of Ganoderma spp. on the growth of Hep G2 and Hep 3B
cells after 48 hr lovastatin treatment.

| | | Cell viability | | |
|---|---|---|---|---|
| Lovastatin | GL-M1 | (% of control) | | |
| (μM) | (μg/ml) | Hep G2 | Hep 3B | Ratio |
| 0 | 0 | 100.0 ± 8.6^{a} | 100.0 ± 2.5^{a} | 1.0 |
| 0 | 10 | 103.2 ± 4.4^{a} | 107.8 ± 8.7^{a} | — |
| 0 | 15 | 116.7 ± 4.2^{b} | 108.5 ± 10.8^{a} | — |
| 0 | 20 | 120.2 ± 8.8^{b} | 73.9 ± 6.2^{b} | — |
| 10 | 0 | 76.9 ± 2.6^{c} | 53.6 ± 8.2^{c} | 2.0** |
| 10 | 10 | 86.2 ± 5.5^{c} | 15.0 ± 4.5^{d} | 6.2*** |
| 10 | 15 | 80.5 ± 4.9^{c} | 0.0 ± 11.4^{d} | 5.1*** |
| 10 | 20 | 82.1 ± 1.0^{c} | 0.0 ± 9.5^{d} | 5.6*** |

1. Cells (5 × 10³/well) were seeded in complete DMEM on 96 well culture plates for 24 hr. They were subsequently incubated in serum-free DMEM and treated with 0 μM and 10 μM lovastatin. Following 48 hr incubation. cells were changed to fresh DMEM containing concentrations of methanolic extracts of Ganoderma spp.
(GL-M1) for another 48 hr. Cell viability was determined by using the MTT assay. Each data represents mean ± SD (n = 6) from the same experiment.
2. Ratio, inhibition (%) on the growth of Hep 3B inhibition (%) on the growth of Hep G2.
*p<0.05,
**p<0.01,
***p<0.001.
3. Data with different superscripts in the column were significantly different by using Student's t-test (p<0.05).

TABLE 23
Effects of combined treatment of lovastatin and caffeic acid
phenethyl ester on the growth of Hep G2 and Hep 3B cells.

| | | Cell viability | | |
|---|---|---|---|---|
| Lovastatin | CAPE | (% of control) | | |
| (μM) | (μM) | Hep G2 | Hep 3B | Ratio |
| 0 | 0 | 100.0 ± 3.2^{a} | 100.0 ± 1.5^{a} | 1.0 |
| 0 | 2.5 | 67.4 ± 8.9^{b} | 81.7 ± 5.6^{b} | 0.6 |
| 0 | 5 | 36.8 ± 6.7^{c} | 74.2 ± 6.8^{b} | 0.4** |
| 0 | 10 | 20.0 ± 4.0^{d} | 53.5 ± 5.0^{c} | 0.6*** |
| 0 | 25 | 13.2 ± 1.1^{e} | 22.3 ± 5.7^{d} | 0.9 |
| 5 | 0 | 66.8 ± 1.5 | 60.7 ± 2.4 | 1.2 |
| 5 | 2.5 | 45.4 ± 3.5 | 52.3 ± 6.9 | 0.9 |
| 5 | 5 | 30.0 ± 3.4 | 52.7 ± 5.9 | 0.7 |
| 5 | 10 | 18.9 ± 1.5 | 40.7 ± 1.5 | 0.7 |
| 5 | 25 | 5.7 ± 1.4 | 19.4 ± 0.9 | 0.9 |
| 10 | 0 | 57.8 ± 11.2^{bf} | 48.5 ± 2.5^{ce} | 1.2 |
| 10 | 2.5 | 40.2 ± 2.2^{cf} | 45.3 ± 1.0^{ef} | 0.9* |
| 10 | 5 | 29.0 ± 1.8^{cg} | 43.3 ± 0.8ⁱ | 0.8*** |
| 10 | 10 | 14.0 ± 1.5^{deh} | 29.6 ± 1.8^{dg} | 0.8*** |
| 10 | 25 | 0.0 ± 2.9ⁱ | 13.3 ± 3.6^{dh} | 0.9*** |

1. Cells (5 × 10³/well) were seeded in complete DMEM on 96 well culture plates for 24 hr. They were subsequently incubated in serum-free DMEM and simultaneously treated with lovastatin and caffeic acid phenethyl ester (CAPE) for 72 hr. Cell viability was determined by using the MTT assay. Each data represents mean ± SD (n = 6) from the same experiment.
2. Ratio, inhibition (%) on the growth of Hep 3B inhibition (%) on the growth of Hep G2. *p < 0.05. **p < 0.01. ***p < 0.001
3. Data with different superscripts in the column were significantly different by using Student's t-test (p < 0.05).

TABLE 24
Effects of caffeic acid phenethyl ester on the growth of
Hep G2 and Hep 3B cells after 48 hr lovastatin treatment.

| | | Cell viability | | |
|---|---|---|---|---|
| Lovastatin | CAPE | (% of control) | | |
| (μM) | (μM) | Hep G2 | Hep 3B | Ratio |
| 0 | 0 | 100.0 ± 8.3^{a} | 100.0 ± 2.6^{a} | 1.0 |
| 0 | 2.5 | 95.3 ± 3.1^{a} | 96.1 ± 4.1^{b} | 0.8 |
| 0 | 5 | 70.2 ± 1.7^{b} | 88.8 ± 1.6^{b} | 0.4*** |
| 0 | 10 | 47.7 ± 5.6^{c} | 77.4 ± 1.8^{c} | 0.4*** |
| 5 | 0 | 75.2 ± 1.6 | 64.3 ± 5.5 | 1.4 |
| 5 | 2.5 | 62.0 ± 1.7 | 73.8 ± 1.9 | 0.7 |
| 5 | 5 | 40.9 ± 1.2 | 70.4 ± 1.1 | 0.5 |
| 5 | 10 | 27.5 ± 1.0 | 54.6 ± 1.8 | 0.5 |
| 10 | 0 | 66.7 ± 2.4^{b} | 59.3 ± 4.5^{d} | 1.2 |
| 10 | 2.5 | 57.8 ± 2.1^{d} | 69.0 ± 3.1^{e} | 0.7*** |
| 10 | 5 | 32.5 ± 2.6^{e} | 55.5 ± 2.5^{d} | 0.7*** |
| 10 | 10 | 22.7 ± 1.1^{f} | 50.0 ± 3.8^{d} | 0.6*** |

1. Cells (5 × 10³/well) were seeded in complete DMEM on 96 well culture plates for 24 hr. They were subsequently incubated in serum-free DMEM and treated with various concentrations of lovastatin. Following 48 hr incubation, cells were changed to fresh DMEM containing various concentrations of caffeic acid phenethyl ester (CAPE) for another 48 hr. Cell viability was determined by using the MTT assay. Each data represents mean ± SD (n = 6) from the same experiment.
2. Ratio, inhibition (%) on the growth of Hep 3B / inhibition (%) on the growth of Hep G2. *p < 0.05. **p < 0.01. ***p < 0.001
3. Data with different superscripts in the column were significantly different by using Student's t-test (p < 0.05).

TABLE 25
Effects of combined treatment of lovastatin and caffeic acid
1-octyl ester on the growth of Hep G2 and Hep 3B cells.

| | | Cell viability | | |
|---|---|---|---|---|
| Lovastatin | CAO | (% of control) | | |
| (μM) | (μM) | Hep G2 | Hep 3B | Ratio |
| 0 | 0 | 100.0 ± 4.1^{a} | 100.0 ± 4.5^{a} | 1.0 |
| 0 | 1.25 | 74.0 ± 2.9^{b} | 66.9 ± 2.9^{b} | 1.3* |
| 0 | 2.5 | 42.5 ± 1.5^{c} | 50.9 ± 0.9^{c} | 0.8** |
| 0 | 5 | 16.2 ± 1.2^{d} | 20.1 ± 5.7^{d} | 0.9 |
| 10 | 0 | 50.3 ± 4.9^{c} | 28.6 ± 1.7^{e} | 1.4** |
| 10 | 1.25 | 69.0 ± 5.0^{b} | 30.4 ± 3.0^{e} | 2.2*** |
| 10 | 2.5 | 51.3 ± 4.5^{e} | 24.3 ± 1.0^{d} | 1.6*** |
| 10 | 5 | 28.3 ± 7.2^{f} | 18.6 ± 6.6^{d} | 1.1** |

1. Cells (5 × 10³/well) were seeded in complete DMEM on 96 well culture plates for 24 hr. They were subsequently incubated in serum-free DMEM and simultaneously treated with lovastatin and CAO for 96 hr. Cell viability was determined by using the MTT assay. Each data represents mean ± SD (n = 6) from the same experiment.
2. Ratio, inhibition on the growth of Hep 3B (%) inhibition on the growth of Hep G2 (%). *p < 0.05, **p < 0.01, ***p < 0.001.
3. Data with different superscripts in the column were significantly different by using Student's t-test (p < 0.05).

TABLE 26
Effects of caffeic acid 1-octyl ester on the growth of Hep
G2 and Hep 3B cells after 48 hr lovastatin treatment.

| | | Cell viability | | |
|---|---|---|---|---|
| Lovastatin | CAO | (% of control) | | |
| (μM) | (μM) | Hep G2 | Hep 3B | Ratio |
| 0 | 0 | 100.0 ± 1.7^{a} | 100.0 ± 4.1^{a} | 1.0 |
| 0 | 1.25 | 99.0 ± 4.1^{a} | 83.0 ± 4.4^{b} | 17.0*** |
| 0 | 2.5 | 51.0 ± 6.0^{b} | 72.1 ± 2.2^{c} | 0.5** |
| 0 | 5 | 42.8 ± 3.0^{b} | 53.2 ± 4.5^{d} | 0.8* |
| 10 | 0 | 75.0 ± 3.3^{c} | 47.9 ± 6.1^{de} | 2.1** |
| 10 | 1.25 | 51.4 ± 6.3^{b} | 34.4 ± 9.1^{ef} | 1.3* |
| 10 | 2.5 | 2.1 ± 12.2^{d} | 31.5 ± 3.6^{f} | 0.7* |
| 10 | 5 | 0.0 ± 6.3^{d} | 15.4 ± 4.5^{g} | 0.8* |

1. Cells (5 × 10³/well) were seeded in complete DMEM on 96 well culture plates for 24 hr. They were subsequently incubated in serum-free DMEM and treated with various concentrations of lovastatin. Following 48 hr incubation, cells were changed to fresh DMEM containing various concentrations of caffeic acid 1-octyl ester (CAO) for another 48 hr. Cell viability was determined by using the MTT assay. Each data represents mean ± SD (n = 6) from the same experiment.
2. Ratio, inhibition (%) on the growth of Hep 3B inhibition (%) on the growth of Hep G2. *p < 0.05. **p < 0.01. ***p < 0.001
3. Data with different superscripts in the column were significantly different by using Student's t-test (p < 0.05).

TABLE 27
Effects of lovastatin and taxol on the tumor growth of Hep 3B/T2 cells in nude mice.

| | Treatment | Tumor size (1 × w² × 0.52 cm³) | | | | | | | |
|---|---|---|---|---|---|---|---|---|---|
| Group | period | Day 8 | Day 11 | Day 15 | Day 17 | Day 20 | Day 23 | Day 26 | Day 29 |
| Control | — | 0.111 ± | 0.125 ± | 0.147 ± | 0.158 ± | 0.170 ± | 0.201 ± | 0.243 ± | 0.381 ± |
| (n = 3) | | 0.026 | 0.038^{d} | 0.085 | 0.092 | 0.091^{a} | 0.175 | 0.325 | 0.539 |
| LOV | 8˜14 | 0.111 ± | 0.048 ± | 0.050 ± | 0.050 ± | 0.051 ± | 0.059 ± | 0.121 ± | 0.154 ± |
| (n = 3) | | 0.026 | 0.021^{b} | 0.046 | 0.043 | 0.046^{a} | 0.032 | 0.140 | 0.097 |
| Taxol | 15˜19 | 0.111 ± | 0.125 ± | 0.125 ± | 0.063 ± | 0.047 ± | 0.037 ± | 0.064 ± | 0.137 ± |
| (n = 3) | | 0.026 | 0.038^{a} | 0.008 | 0.011 | 0.007^{a} | 0.01 | 0.050 | 0.143 |
| LOV | 8˜14 | 0.111 ± | 0.048 ± | 0.046 ± | 0.008 ± | 0.003 ± | 0.014 ± | 0.019 ± | 0.040 ± |
| Taxol | 15˜19 | 0.026 | 0.021^{b} | 0.010 | 0.014 | 0.005^{b} | 0.023 | 0.008 | 0.021 |
| (n = 3) | | | | | | | | | |

1. Male BALB/c-nu/nu nude mice were inoculated with 1 × 10⁷ hepatoma Hep 3B/T2 cells on day 0. Mice were treated on the indicated days after tumor inoculation by p.o. lovastatin (LOV, 75 mg/kg/day) for 7 days and/or i.p. taxol (120 μg/kg/day) for 5 days. Measurement of tumor size was started on day 8 after inoculation. Each data represents mean ± SEM (n = 3) of the same experiment.
2. Treatment period were indicated the days after tumor inoculation.
3. Data with different superscripts in the same column were significantly different by using one-way ANOVA (p < 0.05).

TABLE 28
Effects of lovastatin and taxol on the tumor growth of Hep 3B/T2 cells in nude mice.

| | Treatment | Tumor size (1 × w² × 0.52 cm³) | | | | | | |
|---|---|---|---|---|---|---|---|---|
| Group | period | Day 28 | Day 35 | Day 38 | Day 42 | Day 49 | Day 56 | Day 63 |
| Control | — | 0.170 ± | 0.400 ± | 0.690 ± | 0.919 ± | 1.387 ± | 1.860 ± | 2.503 ± |
| (n = 6) | | 0.100 | 0.120 | 0.180^{d} | 0.179^{a} | 1.063 | 1.186 | 1.848^{a} |
| LOV | 28˜34, 49˜55 | 0.170 ± | 0.200 ± | 0.301 ± | 0.533 ± | 0.665 ± | 0.995 ± | 1.263 ± |
| (n = 7) | | 0.080 | 0.180 | 0.224^{b} | 0.355^{d} | 0.962 | 1.491 | 1.820^{ab} |
| Taxol | 36, 38, 40, | 0.170 ± | 0.400 ± | 0.415 ± | 0.465 ± | 0.811 ± | 0.996 ± | 1.424 ± |
| (n = 7) | 57, 59, 61 | 0.100 | 0.140 | 0.190^{a} | 0.209^{a} | 0.584 | 0.745 | 0.933^{a} |
| LOV + | 28˜34, 49˜55 | 0.170 ± | 0.200 ± | 0.171 ± | 0.109 ± | 0.144 ± | 0.171 ± | 0.321 ± |
| Taxol | 36, 38, 40, | 0.080 | 0.180 | 0.145^{b} | 0.179^{b} | 0.157 | 0.239 | 0.456^{b} |
| (n = 7) | 57, 59, 61 | | | | | | | |

1. Male BALB/c-nu/nu nude mice were inoculated with 1 × 10⁷ hepatoma Hep 3B/T2 cells on day 0. Mice were treated on the indicated days after tumor inoculation by p.o. lovastatin (LOV, 37.5 mg/kg/day) for 14 days and/or i.p. taxol (120 μg/kg/2 day) for day 36, 38, 40, 57, 59, and 61. Measurement of tumor size was started on day 28 after inoculation. Each data represents mean ± SEM of the same experiment.
2. Treatment period were indicated the days after tumor inoculation.
3. Data with different superscripts in the same column were significantly different by using one-way ANOVA (p < 0.05)

TABLE 29
Effect of lovastatin and taxol on the tumor
growth of Hep 3B/T2 cells in nude mice.

| | | Tumor size | | | |
|---|---|---|---|---|---|
| | | (1 × w² × 0.52 cm³) | | | |
| | | Cycle 1 | | Cycle 2 | |
| Group | | (Day 42) | | (Day 63) | |
| Control | | 0.919 ± | | 2.503 ± | |
| | | 0.179^{a} | | 1.848^{a*} | |
| | (n = 6) | | | | |
| LOV | | 0.533 ± | (−43.6 ± | 1.263 ± | (−49.5 ± |
| | | 0.355^{a} | 74.6) | 1.820^{ab} | 57.5) |
| | (n = 7) | | | | |
| Taxol | | 0.465 ± | (−54.2 ± | 1.424 ± | (−43.1 ± |
| | | 0.209^{a} | 29.1) | 0.933^{a*} | 31.1) |
| | (n = 7) | | | | |
| LOV + | | 0.109 ± | (−89.4 ± | 0.321 ± | (−87.2 ± |
| Taxol | | 0.179^{b} | 11.3) | 0.456^{b} | 15.2) |
| | (n = 7) | | | | |

1. Male BALB/c-nu/nu nude mice were inoculated with 1 × 10⁷ hepatoma Hep 3B/T2 cells on day 0. Mice were treated on the indicated days after tumor inoculation by p.o. lovastatin (LOV, 37.5 mg/kg/day) for days 28-34 and 49-55 and/or i.p. taxol (120 μg/kg/2 day) for day 36, 38, 40, 57, 59, and 61. Measurement of tumor size was started on day 28 after inoculation. Each data represents mean ± SEM of the same experiment.
2. The percentages of inhibition were shown in parentheses.
3. Data with different superscripts in the same column were significantly different by using one-way ANOVA (p < 0.05).
4. Value with the * at day 63 was significantly different as compared with day 42.

1. A method for treating cancer cells in a mammal, comprising the steps of:
(a) sensitizing said cancer cells by administering to the mammal an agent which comprises an effective amount of a HMG-CoA reductase inhibitor; and
(b) treating the sensitized cancer cells with an anti-cancer agent which reduces the survival or growth of the cancer cells.

2. The method of claim 1, wherein said anti-cancer agent causes apoptosis through arresting the growth of the cancer cells at the G2/M stage.

3. The method of claim 1, wherein said HMC-CoA reductase inhibitor is lovastatin.

4. The method of claim 2, wherein said HMC-CoA reductase inhibitor is lovastatin.

5. The method of claim 4, wherein said cancer cells are in the liver of a mammal.

6. The method of claim 5, wherein said cancer cells are in a human liver.

7. The method of claim 1, wherein said anti-cancer agent comprises paclitaxel.

8. The method of claim 1, wherein said anti-cancer agent comprises CAPE.

9. The method of claim 1, wherein said anti-cancer agent comprises Ganoderma spp. extract.

10. A nutraceutical composition which comprises oxygenated triterpenes extracted from pulverized Ganoderma fruiting bodies with methanol or a combination of ethanol and water.

11. The nutraceutical composition of claim 10 wherein the extracted oxygenated triterpenes can be eluted from a Cosmosil 5C18, 250×8 mm HPLC column with methanol:water:acetic acid=70:30:0.5 (V:V:V) at 2.0 ml/min and collected between 4 and 22 minutes.

12. A method of preparing a nutraceutical composition comprising oxygenated triterpenes comprising the step of extracting from pulverized Ganoderma fruiting bodies with methanol or a combination of ethanol and water at a elevated temperature to form a crude extract.

13. The method of claim 12 further comprising the step of solvent partitioning or reverse phase chromatography so as to obtain the oxygenated triterpenes in an enriched form.

14. A method of treating cancer by administrating to a patient in need thereof a nutraceutical composition of claim 10.

15. The method of claim 14, wherein said cancer is liver cancer.

16. The method of claim 9, wherein said Ganoderma ssp extract is extracted from pulverized Ganoderma fruiting bodies with methanol or a combination of ethanol and water.

17. The method of claim 16, wherein said Ganoderma ssp extract can be eluted from a Cosmosil 5C18, 250×8 mm HPLC column with methanol:water:acetic acid 70:30:0.5 (V:V:V) at 2.0 ml/min and collected between 4 and 22 minutes.

18. The method of treating cancer by administering to a patient in Deed thereof a nutraceutical composition according to claim 11.

19. A method of treating a patient with cancer, comprising the steps of:
(a) administrating to the patient a first agent which arrests the growth of cancer cells at a specific stage in the cell cycle to sensitize said cancel cells; and
(b) administrating to the patient a second agent which can arrest the growth of said sensitized cancer cells at a later time point in the cell cycle as compared to the first agent.

20. The method of claim 19, wherein the cancer is liver cancer.

21. The method of claim 19, wherein said first agent arrests die growth of cancer cells at stage G0/G1 of the cell cycle and said second agent arrests the growth of cancer cells at stage G2/M of the cell cycle.

22. The method of claim 19, wherein said first and second agents arrest the growth of cancer cells at the same cell cycle stage.
